(19) United States
(12) Patent Application Publication
(10) Pub. No.: US 2025/0017549 A1
(43) Pub. Date: Jan. 16, 2025

(54) AUTOMATED SCANNING SYSTEM AND METHOD

(71) Applicant: SHANGHAI UNITED IMAGING HEALTHCARE CO., LTD., Shanghai (CN)

(72) Inventors: Xuan LIU, Shanghai (CN); Yifeng JIANG, Shanghai (CN); Pei ZHOU, Shanghai (CN); Yangyang LIN, Shanghai (CN)

(73) Assignee: SHANGHAI UNITED IMAGING HEALTHCARE CO., LTD., Shanghai (CN)

(21) Appl. No.: 18/898,664

(22) Filed: Sep. 26, 2024

Related U.S. Application Data
(63) Continuation of application No. PCT/CN2022/084949, filed on Apr. 2, 2022.

(51) Int. Cl. A61B 6/00; A61B 6/03; G06T 7/20; G06T 7/70

(52) U.S. Cl. CPC A61B 6/547; A61B 6/545; A61B 6/56; G06T 7/20; G06T 7/70; A61B 6/032; G06T 2207/30004

(57) ABSTRACT

The present disclosure provides an automated scanning system and method. The method may include obtaining region information of a region. The method may also include determining, based on the region information, a route from a start position of a scanning apparatus to a target position. The method may further include causing the scanning apparatus to move to the target position along the route. The method may still further include identifying a target portion of a subject based on image data obtained from at least one optical device set on the scanning apparatus, and causing the scanning apparatus to scan the target portion of the subject.

### CROSS REFERENCE TO RELATED APPLICATIONS

This application is a continuation of International Application No. PCT/CN2022/084949, filled on Apr. 2, 2022, the entire contents of which is incorporated herein by reference.

### TECHNICAL FIELD

The present disclosure generally relates to medical imaging and/or treatment, and in particular, to systems and methods for automated scanning of a subject.

### BACKGROUND

Mobile scanning apparatuses (e.g., a mobile computed tomography (CT) scanner) are increasingly widely used in clinical diagnosis and/or treatment. Conventionally, a mobile scanning apparatus may be manually driven to a position where a subject to be scanned by a user. In addition, the mobile scanning apparatus may be charged through wire cables. The motion and/or operation of the mobile scanning apparatus under manpower may be inefficient and toilsome. Also, the wire cables may need to be connected to the mobile scanning apparatus by the user for charging the mobile scanning apparatus. The user may trip over the wire cables during the process. Therefore, it is desirable to provide systems and methods for automated scanning of a subject without manpower.

### SUMMARY

According to an aspect of the present disclosure, a system is provided. The system may comprise at least one storage medium including a set of instructions; and at least one processor configured to communicate with the at least one storage medium, wherein when executing the set of instructions, the at least one processor is configured to direct the system to perform operations. The operations may include obtaining region information of a region; determining, based on the region information, a route from a start position of a scanning apparatus to a target position; causing the scanning apparatus to move to the target position along the route; identifying a target portion of a subject based on image data obtained from at least one optical device set on the scanning apparatus; and causing the scanning apparatus to scan the target portion of the subject.

According to another aspect of the present disclosure, a method implemented on a computing device having a processor and a computer-readable storage device is provided. The method may include obtaining region information of a region; determining, based on the region information, a route from a start position of a scanning apparatus to a target position; causing the scanning apparatus to move to the target position along the route; identifying a target portion of a subject based on image data obtained from at least one optical device set on the scanning apparatus; and causing the scanning apparatus to scan the target portion of the subject.

According to a further aspect of the present disclosure, a non-transitory readable medium including at least one set of instructions is provided. When executed by at least one processor of a computing device, the at least one set of instructions may direct the at least one processor to perform a method. The method may include obtaining region information of a region; determining, based on the region information, a route from a start position of a scanning apparatus to a target position; causing the scanning apparatus to move to the target position along the route; identifying a target portion of a subject based on image data obtained from at least one optical device set on the scanning apparatus; and causing the scanning apparatus to scan the target portion of the subject.

In some embodiments, the obtaining region information of a region includes: obtaining sensing data from one or more sensors set on the scanning apparatus; and generating, based on the sensing data, the region information of the region.

In some embodiments, the one or more sensors include at least one distance sensor and at least one second optical device.

In some embodiments, the region information includes map data of the region.

In some embodiments, the at least one distance sensor includes a first distance sensor and a second distance sensor, and the generating, based on the sensing data, region information of a region includes: determining environmental information of the region based on sensing data from the first distance sensor; determining first supplementary information of the region based on sensing data from the at least one second optical device; determining second supplementary information of the region based on sensing data from the second distance sensor; and generating the map data of the region based on the environmental information, the first supplementary information, and the second supplementary information.

In some embodiments, the region information further includes route condition information.

In some embodiments, the generating, based on the sensing data, region information of a region includes: identifying moving objects in the region based on the sensing data from the at least one second optical device; determining movement statuses of the moving objects; and generating the route condition information based on the movement statuses of the moving objects.

In some embodiments, the causing the scanning apparatus to move to the target position along the route includes: determining a moving speed of the scanning apparatus based on the route condition information; and causing the scanning apparatus to move to the target position along the route at the determined moving speed.

In some embodiments, the region information includes map data of the region.

In some embodiments, the determining, based on the region information, a route from a start position of a scanning apparatus to a target position where a subject is located includes: obtaining a moving model; and determining the route from the start position of the scanning apparatus to the target position by inputting the region information, the start position, and the target position into the moving model.

In some embodiments, the moving model is a machine learning model.

In some embodiments, the causing the scanning apparatus to scan the target portion of the subject includes: obtaining at least one of a position difference or a direction difference between the target portion and the scanning apparatus; and causing the scanning apparatus to adjust at least one of a position or a direction of the scanning apparatus according to at least one of the position difference or the direction difference.

In some embodiments, the operations further include determining a moving mode of the scanning apparatus according to at least one of the region information or user instructions.

In some embodiments, the determination of the route from the start position to the target position and the moving of the scanning apparatus to the target position along the route are in accordance with the moving mode.

In some embodiments, the operations further include causing the scanning apparatus to move to a charging area for charging the scanning apparatus at a contactless charging station or a contact charging station in the charging area.

In some embodiments, the contactless charging station includes a plurality of wireless power transmitters set in a transmitting area, the plurality of wireless power transmitters being electrically connected to a power source; the scanning apparatus includes a wireless power receiver and a power storage device, the wireless power receiver being electrically connected to the power storage device; and the wireless power receiver is operably connected to one of the plurality of wireless power transmitters wirelessly to facilitate the charging of the power storage device of the scanning apparatus when the scanning apparatus is located in the charging area.

In some embodiments, the one of the plurality of wireless power transmitters includes a power transmitting coil, the wireless power receiver includes a power receiving coil, and the power transmitting coil is aligned with the power receiving coil when the wireless power receiver is operably connected to the one of the plurality of wireless power transmitters wirelessly.

In some embodiments, the power receiving coil is driven by a coil adjustment device to move to be aligned with the power transmitting coil.

In some embodiments, the transmitting area is within the charging area or in a plane at an angle with the charging area.

In some embodiments, the scanning apparatus further includes a monitoring device configured to determine a status of the power storage device during the charging.

In some embodiments, the status of the power storage device relates to at least one of a temperature, a voltage value, or a current value of the power storage device.

In some embodiments, the operations further include determining an updated route from an intermediate position between the start position and the target position to the target position.

In some embodiments, the scanning apparatus includes a computed tomography (CT) scanner.

According to a still further aspect of the present disclosure, a system is provided. The system may comprise a scanning apparatus, configured to scan a target portion of a subject; at least one optical device, configured to generate image data including a representation of the subject; at least one processor, configured to: obtain region information of a region; determine, based on the region information, a route from a start position of a scanning apparatus to a target position; cause the scanning apparatus to move to the target position along the route; and identify the target portion of the subject based on the image data including the representation of the subject.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary automated scanning system according to some embodiments of the present disclosure;

FIG. 2 is a schematic diagram illustrating an exemplary hardware and/or software components of an exemplary computing device according to some embodiments of the present disclosure;

FIG. 3 is a schematic diagram illustrating hardware and/or software components of an exemplary mobile device according to some embodiments of the present disclosure;

FIG. 4A is a block diagram illustrating an exemplary processing device according to some embodiments of the present disclosure;

FIG. 4B is a block diagram illustrating an exemplary power module of the scanning apparatus according to some embodiments of the present disclosure;

FIG. 5 is a flowchart illustrating an exemplary process for causing a scanning apparatus to scan a target portion of a subject automatically according to some embodiments of the present disclosure;

FIG. 6 is a flowchart illustrating an exemplary process for generating map data of a region according to some embodiments of the present disclosure;

FIG. 7 is a schematic diagram illustrating automated scanning of a target portion of a subject according to some embodiments of the present disclosure;

FIG. 8 is a flowchart illustrating an exemplary process for initiating wireless charging of the scanning apparatus according to some embodiments of the present disclosure;

FIG. 9 is a schematic diagram illustrating wireless charging of the scanning apparatus according to some embodiments of the present disclosure; and

FIGS. 10A and 10B are schematic diagrams illustrating exemplary configurations of a wireless power receiver and a plurality of wireless power transmitters for wireless charging of a scanning apparatus according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the terms “system,” “engine,” “unit,” “module,” and/or “block” used herein are one method to distinguish different components, elements, parts, sections, or assemblies of different levels in ascending order. However, the terms may be displaced by another expression if they achieve the same purpose.

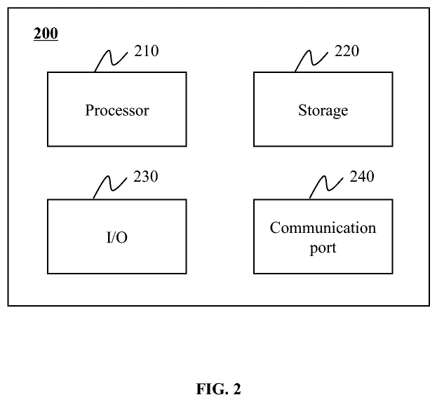

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processor 210 as illustrated in FIG. 2) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments of the present disclosure. It is to be expressly understood the operations of the flowcharts may be implemented not in order. Conversely, the operations may be implemented in an inverted order, or simultaneously. Moreover, one or more other operations may be added to the flowcharts. One or more operations may be removed from the flowcharts.

Provided herein are systems and methods for non-invasive imaging, such as for disease diagnosis, treatment, and/or research purposes. In some embodiments, the automated scanning system may include a single modality system and/or a multi-modality system. The term “modality” used herein broadly refers to an imaging or treatment method or technology that gathers, generates, processes, and/or analyzes imaging information of a subject or treatments the subject. The single modality system may include a computed tomography (CT) system, a magnetic resonance imaging (MRI) system, an ultrasound automated scanning system, an X-ray automated scanning system, an ultrasonography system, a positron emission tomography (PET) system, an optical coherence tomography (OCT) automated scanning system, an ultrasound (US) automated scanning system, an intravascular ultrasound (IVUS) automated scanning system, a near-infrared spectroscopy (NIRS) automated scanning system, or the like, or any combination thereof. The multi-modality system may include an X-ray imaging-magnetic resonance imaging (X-ray-MRI) system, a positron emission tomography-X-ray imaging (PET-X-ray) system, a single-photon emission computed tomography-magnetic resonance imaging (SPECT-MRI) system, a positron emission tomography-computed tomography (PET-CT) system, a C-arm system, a positron emission tomography-magnetic resonance imaging (PET-MR) system, a digital subtraction angiography-magnetic resonance imaging (DSA-MRI) system, or the like, or any combination thereof.

In the present disclosure, the term “image” may refer to a two-dimensional (2D) image, a three-dimensional (3D) image, or a four-dimensional (0) image. In some embodiments, the term “image” may refer to an image of a region (e.g., a region of interest (ROI)) of a subject. As described above, the image may be a CT image, a PET image, an MR image, a fluoroscopy image, an ultrasound image, an Electronic Portal Imaging Device (EPID) image, etc.

As used herein, a representation of a subject (e.g., a patient, or a portion thereof) in an image may be referred to as the subject for brevity. For instance, a representation of an organ or tissue (e.g., the heart, the liver, a lung, etc., of a patient) in an image may be referred to as the organ or tissue for brevity. An image including a representation of a subject may be referred to as an image of the subject or an image including the subject for brevity. As used herein, an operation on a representation of a subject in an image may be referred to as an operation on the subject for brevity. For instance, a segmentation of a portion of an image including a representation of an organ or tissue (e.g., the heart, the liver, a lung, etc., of a patient) from the image may be referred to as a segmentation of the organ or tissue for brevity.

An aspect of the present disclosure relates to systems and methods for automated scanning of a subject. The system may obtain region information of a region (e.g., a hospital) and determine, based on the region information, a route from a start position of a scanning apparatus to a target position where a subject is located. The system may cause the scanning apparatus to move to the target position along the route. After the scanning apparatus arrives at the target position, a target portion of the subject may be identified based on image data obtained from at least one optical device set on the scanning apparatus. The system may cause the scanning apparatus to adjust its position and scan the target portion of the subject. In addition, after the scanning of the target portion is complete, the system may further cause the scanning apparatus to move to a charging area for wireless charging. The entire process may be controlled by the system automatically, thus saving manpower and improving the efficiency of the scanning procedure.

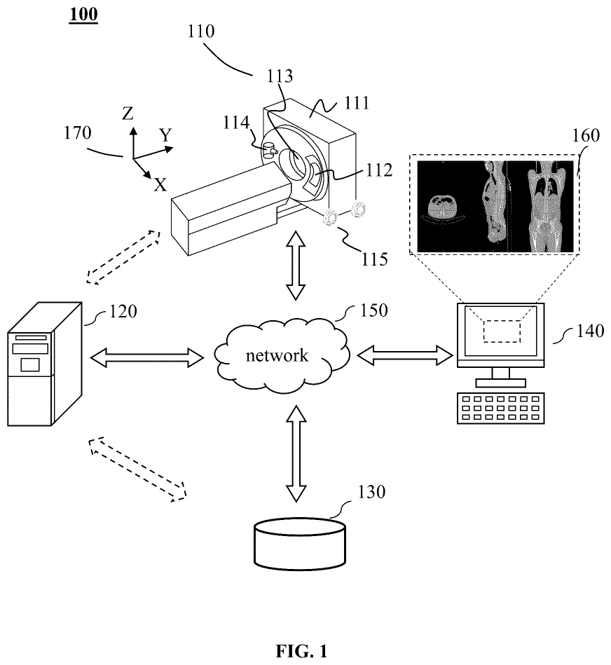

FIG. 1 is a schematic diagram illustrating an exemplary automated scanning system according to some embodiments of the present disclosure. As illustrated in FIG. 1, the automated scanning system 100 may include a scanning apparatus 110, a processing device 120, a storage device 130, a terminal device 140, and a network 150. In some embodiments, two or more components of the automated scanning system 100 may be connected to and/or communicate with each other via a wireless connection, a wired connection, or a combination thereof. The connection among the components of the automated scanning system 100 may be variable. Merely by way of example, the scanning apparatus 110 may be connected to the processing device 120 through the network 150 or directly. As another example, the storage device 130 may be connected to the processing device 120 through the network 150 or directly.

The scanning apparatus 110 may be configured to scan a subject or a portion thereof that is located within its detecting region and generate scanning data/signals relating to the (portion of) subject. The scanning apparatus 110 may be a mobile scanning apparatus.

In some embodiments, the scanning apparatus 110 may include a single modality device. For example, the scanning apparatus 110 may include a CT scanner, a PET scanner, a SPECT scanner, an MR scanner, an ultrasonic scanner, an ECT scanner, or the like, or a combination thereof. In some embodiment, the scanning apparatus 110 may be a multi-modality device. For example, the scanning apparatus 110 may include a PET-CT scanner, a PET-MR scanner, or the like, or a combination thereof. The following descriptions are provided, unless otherwise stated expressly, with reference to a CT scanner for illustration purposes and not intended to be limiting.

As illustrated, the CT scanner may include a gantry 111, a detector 112, a detecting region 113, a radiation source 114, and a driving device 115. The gantry 111 may support the detector 112 and the radiation source 114. The driving device 115 may include a driver and a motion mechanism (e.g., wheels, a pedrail, etc.). The driving device 115 may drive the scanning apparatus 110 to move to any position (e.g., a position where the subject is located, a charging area, etc.) in a region. After the driving device 115 drives the scanning apparatus 110 to the position where the subject is located, the subject or a portion thereof may be placed in the detecting region 113 for scanning. The radiation source 114 may emit x-rays. The x-rays may be emitted from a focal spot using a high-intensity magnetic field to form an x-ray beam. The x-ray beam may travel toward the subject or the portion thereof. The detector 112 may detect x-ray photons from the detecting region 113. In some embodiments, the detector 112 may include one or more detector units. The detector unit(s) may be and/or include single-row detector elements and/or multi-row detector elements.

The processing device 120 may process data and/or information. The data and/or information may be obtained from the scanning apparatus 110 or retrieved from the storage device 130, the terminal device 140, and/or an external device (external to the automated scanning system 100) via the network 150. For example, the processing device 120 may reconstruct map data of the region based on sensing data from one or more sensors set on the scanning apparatus 110. As another example, the processing device 120 may determine a route from a start position of the scanning apparatus 110 to the target position where the subject is located. As a further example, the processing device 120 may identify a target portion of the subject and cause the scanning apparatus 110 to scan the target portion. As still a further example, the processing device 120 may cause the scanning apparatus 110 to move to a charging area for wireless charging of the scanning apparatus 110 after the scanning of the target portion is complete. In some embodiments, the processing device 120 may be a single server or a server group. The server group may be centralized or distributed. In some embodiments, the processing device 120 may be local or remote. For example, the processing device 120 may access information and/or data stored in the scanning apparatus 110, the terminal device 140, and/or the storage device 130 via the network 150. As another example, the processing device 120 may be directly connected to the scanning apparatus 110, the terminal device 140, and/or the storage device 130 to access stored information and/or data. In some embodiments, the processing device 120 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing device 120 may be implemented by a computing device 200 having one or more components as illustrated in FIG. 2.

The storage device 130 may store data, instructions, and/or any other information. In some embodiments, the storage device 130 may store data obtained from the scanning apparatus 110 (e.g., scanning data of the subject, sensing data of the one or more sensors set on the scanning apparatus 110, etc.), the terminal device 140, and/or the processing device 120. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or any combination thereof. Exemplary mass storage may include a magnetic disk, an optical disk, a solid-state drive, etc. Exemplary removable storage may include a flash drive, a floppy disk, an optical disk, a memory card, a zip disk, a magnetic tape, etc. Exemplary volatile read-and-write memory may include a random access memory (RAM). Exemplary RAM may include a dynamic RAM (DRAM), a double date rate synchronous dynamic RAM (DDR SDRAM), a static RAM (SRAM), a thyristor RAM (T-RAM), and a zero-capacitor RAM (Z-RAM), etc. Exemplary ROM may include a mask ROM (MROM), a programmable ROM (PROM), an erasable programmable ROM (EPROM), an electrically erasable programmable ROM (EEPROM), a compact disk ROM (CD-ROM), and a digital versatile disk ROM, etc. In some embodiments, the storage device 130 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 130 may be connected to the network 150 to communicate with one or more other components (e.g., the processing device 120, the terminal device 140) of the automated scanning system 100. One or more components of the automated scanning system 100 may access the data or instructions stored in the storage device 130 via the network 150. In some embodiments, the storage device 130 may be directly connected to or communicate with one or more other components (e.g., the processing device 120, the terminal device 140) of the automated scanning system 100. In some embodiments, the storage device 130 may be part of the processing device 120.

The terminal device 140 may input/output signals, data, information, etc. In some embodiments, the terminal device 140 may enable a user interaction with the processing device 120 and/or the scanning apparatus 110. For example, the terminal device 140 may display an image of the subject on a screen 160. As another example, the terminal device 140 may obtain a user's input information through an input device (e.g., a keyboard, a touch screen, a brain wave monitoring device), and transmit the input information to the processing device 120 and/or the scanning apparatus 110 for further processing. The terminal device 140 may be a mobile device, a tablet computer, a laptop computer, a desktop computer, or the like, or any combination thereof. In some embodiments, the mobile device may include a home device, a wearable device, a virtual reality device, an augmented reality device, or the like, or any combination thereof. The home device may include a lighting device, a control device of an intelligent electrical apparatus, a monitoring device, a television, a video camera, an interphone, or the like, or any combination thereof. The wearable device may include a bracelet, a footgear, eyeglasses, a helmet, a watch, clothing, a backpack, an accessory, or the like, or any combination thereof. The virtual reality device and/or the augmented reality device may include a virtual reality helmet, virtual reality glasses, a virtual reality patch, an augmented reality helmet, augmented reality glasses, an augmented reality patch, or the like, or any combination thereof. For example, the virtual reality device and/or the augmented reality device may include a Google Glass™, an Oculus Rift™, a Hololens™, a Gear VR™ etc. In some embodiments, the terminal device 140 may be part of the processing device 120 or a peripheral device of the processing device 120 (e.g., a console connected to and/or communicating with the processing device 120).

The network 150 may include any suitable network that can facilitate the exchange of information and/or data for the automated scanning system 100. In some embodiments, one or more components (e.g., the scanning apparatus 110, the terminal device 140, the processing device 120, the storage device 130) of the automated scanning system 100 may communicate information and/or data with one or more other components of the automated scanning system 100 via the network 150. The network 150 may be and/or include a public network (e.g., the Internet), a private network (e.g., a local area network (LAN), a wide area network (WAN))), a wired network (e.g., an Ethernet network), a wireless network (e.g., an 802.11 network, a Wi-Fi network), a cellular network (e.g., a Long Term Evolution (LTE) network, 4G network, 5G network), a frame relay network, a virtual private network (VPN), a satellite network, a telephone network, routers, hubs, switches, server computers, and/or any combination thereof. Merely by way of example, the network 150 may include a cable network, a wireline network, a fiber-optic network, a telecommunications network, an intranet, a wireless local area network (WLAN), a metropolitan area network (MAN), a public telephone switched network (PSTN), a Bluetooth™ network, a ZigBee™ network, a near field communication (NFC) network, or the like, or any combination thereof. In some embodiments, the network 150 may include one or more network access points. For example, the network 150 may include wired and/or wireless network access points such as base stations and/or internet exchange points through which one or more components of the automated scanning system 100 may be connected to the network 150 to exchange data and/or information.

For illustration purposes, a coordinate system 170 is provided in FIG. 1. The coordinate system 170 may be a Cartesian system including an X-axis, a Y-axis, and a Z-axis. The X-axis and the Y-axis shown in FIG. 1 may be horizontal and the Z-axis may be vertical. As illustrated, the positive X direction along the X-axis may be from the left side to the right side of a table where the subject is positioned viewed from the direction facing the front of the scanning apparatus 110; the positive Y direction along the Y-axis shown in FIG. 1 may be from the end to the head of the table; the positive Z direction along the Z-axis shown in FIG. 1 may be from the lower part to the upper part of the scanning apparatus 110.

It should be noted that the above description regarding the automated scanning system 100 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the automated scanning system 100 may include one or more additional components and/or one or more components of the automated scanning system 100 described above may be omitted. In some embodiments, a component of the automated scanning system 100 may be implemented on two or more sub-components. Two or more components of the automated scanning system 100 may be integrated into a single component.

FIG. 2 is a schematic diagram illustrating exemplary hardware and/or software components of an exemplary computing device according to some embodiments of the present disclosure. The computing device 200 may be configured to implement any component of the automated scanning system 100. For example, the scanning apparatus 110, the processing device 120, the storage device 130, and/or the terminal device 140 may be implemented on the computing device 200. Although only one such computing device is shown for convenience, the computer functions relating to the automated scanning system 100 as described herein may be implemented in a distributed fashion on a number of similar platforms, to distribute the processing load. As illustrated in FIG. 2, the computing device 200 may include a processor 210, a storage 220, an input/output (I/O) 230, and a communication port 240.

The processor 210 may execute computer instructions (e.g., program codes) and perform functions of the processing device 120 in accordance with techniques described herein. The computer instructions may include, for example, routines, programs, objects, components, signals, data structures, procedures, modules, and functions, which perform particular functions described herein. In some embodiments, the processor 210 may perform instructions obtained from the terminal device 140 and/or the storage device 130. In some embodiments, the processor 210 may include one or more hardware processors, such as a microcontroller, a microprocessor, a reduced instruction set computer (RISC), an application-specific integrated circuits (ASICs), an application-specific instruction-set processor (ASIP), a central processing unit (CPU), a graphics processing unit (GPU), a physics processing unit (PPU), a microcontroller unit, a digital signal processor (DSP), a field-programmable gate array (FPGA), an advanced RISC machine (ARM), a programmable logic device (PLD), any circuit or processor capable of executing one or more functions, or the like, or any combinations thereof.

Merely for illustration, only one processor is described in the computing device 200. However, it should be noted that the computing device 200 in the present disclosure may also include multiple processors. Thus operations and/or method steps that are performed by one processor as described in the present disclosure may also be jointly or separately performed by the multiple processors. For example, if in the present disclosure the processor of the computing device 200 executes both operation A and operation B, it should be understood that operation A and operation B may also be performed by two or more different processors jointly or separately in the computing device 200 (e.g., a first processor executes operation A and a second processor executes operation B, or the first and second processors jointly execute operations A and B).

The storage 220 may store data/information obtained from the scanning apparatus 110, the terminal device 140, the storage device 130, or any other component of the medical system 100. In some embodiments, the storage 220 may include a mass storage device, a removable storage device, a volatile read-and-write memory, a read-only memory (ROM), or the like, or any combination thereof. In some embodiments, the storage 220 may store one or more programs and/or instructions to perform exemplary methods described in the present disclosure.

The I/O 230 may input or output signals, data, and/or information. In some embodiments, the I/O 230 may enable user interaction with the processing device 120. In some embodiments, the I/O 230 may include an input device and an output device. Exemplary input devices may include a keyboard, a mouse, a touch screen, a microphone, a camera capturing gestures, or the like, or a combination thereof. Exemplary output devices may include a display device, a loudspeaker, a printer, a projector, a 3D hologram, a light, a warning light, or the like, or a combination thereof. Exemplary display devices may include a liquid crystal display (LCD), a light-emitting diode (LED)-based display, a flat panel display, a curved screen, a television device, a cathode ray tube (CRT), or the like, or a combination thereof.

The communication port 240 may be connected with a network (e.g., the network 150) to facilitate data communications. The communication port 240 may establish connections between the scanning apparatus 110 and the processing device 120, the terminal device 140, or the storage device 130. The connection may be a wired connection, a wireless connection, or a combination of both that enables data transmission and reception. The wired connection may include an electrical cable, an optical cable, a telephone wire, or the like, or any combination thereof. The wireless connection may include a Bluetooth network, a Wi-Fi network, a WiMax network, a WLAN, a ZigBee network, a mobile network (e.g., 3G, 4G, 5G), or the like, or any combination thereof. In some embodiments, the communication port 240 may be a standardized communication port, such as RS232, RS485, etc. In some embodiments, the communication port 240 may be a specially designed communication port. For example, the communication port 240 may be designed in accordance with the digital imaging and communications in medicine (DICOM) protocol.

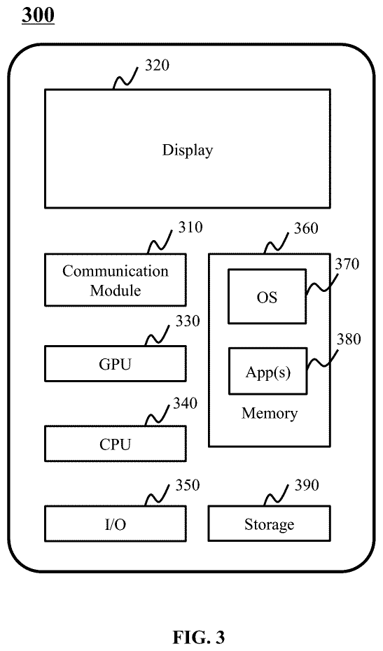

FIG. 3 is a schematic diagram illustrating exemplary hardware and/or software components of an exemplary mobile device according to some embodiments of the present disclosure. In some embodiments, the processing device 120 or the terminal device 140 may be implemented on the mobile device 300. As illustrated in FIG. 3, the mobile device 300 may include a communication module 310, a display 320, a graphics processing unit (GPU) 330, a central processing unit (CPU) 340, an I/O 350, a memory 360, and storage 390. The CPU 340 may include interface circuits and processing circuits similar to the processor 210. In some embodiments, any other suitable component, including but not limited to a system bus or a controller (not shown), may also be included in the mobile device 300. In some embodiments, a mobile operating system 370 (e.g., iOS™, Android™, Windows Phone™) and one or more applications 380 may be loaded into the memory 360 from the storage 390 in order to be executed by the CPU 340. The applications 380 may include a browser or any other suitable mobile apps for receiving and rendering information relating to imaging from the automated scanning system on the mobile device 300. User interactions with the information stream may be achieved via the I/O devices 350 and provided to the processing device 120 and/or other components of the automated scanning system 100 via the network 150.

To implement various modules, units, and their functionalities described in the present disclosure, computer hardware platforms may be used as the hardware platform(s) for one or more of the elements described herein. A computer with user interface elements may be used to implement a personal computer (PC) or any other type of work station or terminal device. A computer may also act as a server if appropriately programmed.

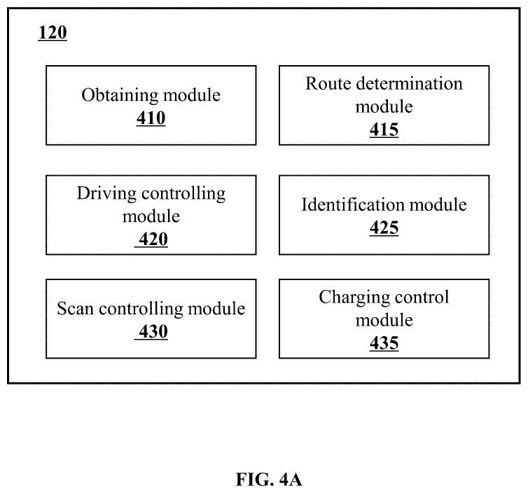

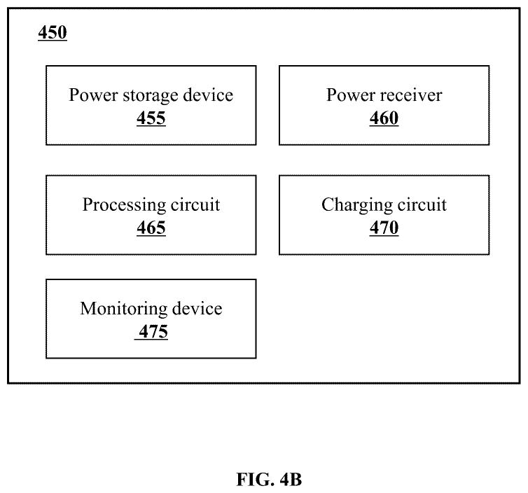

FIG. 4A is a block diagram illustrating an exemplary processing device according to some embodiments of the present disclosure. As illustrated in FIG. 4A, the processing device 120 may include an obtaining module 410, a route determination module 415, a driving controlling module 420, an identification module 425, a scan controlling module 430, and a charging control module 435.

The obtaining module 410 may obtain data or information. The obtaining module 410 may obtain data and/or information from the scanning apparatus 110, the one or more sensors set on the scanning apparatus 110 and/or set at one or more positions in a region, the storage device 130, the terminal(s) 140, or any devices or components capable of storing data via the network 150. In some embodiments, the obtaining module 410 may obtain region information of a region. In some embodiments, the region information may include map data of the region. The map data of the region may include, for example, one or more digital maps of the region. In some embodiments, the map data of the region may be stored in a storage device (e.g., the storage device 130, the storage 220, the storage 309, a cloud storage, etc.). The obtaining module 410 may retrieve the map data of the region from the storage device. In some embodiments, the obtaining module 410 may obtain sensing data from the one or more sensors set on the scanning apparatus 110 and/or set at one or more positions in the region. The one or more sensors may include at least one distance sensor and at least one second optical device. In some embodiments, the region information may also include route condition information. The route condition information may include, for example, a route width, a route length, movement statuses of moving objects, a count of the moving objects, etc., of each route in the region. The moving objects may be, for example, a doctor, a patient, another scanning apparatus, a wheelchair moving in the region, etc.

The route determination module 415 may determine a route from a start position of the scanning apparatus 110 to a target position where a subject is located. In some embodiments, the route determination module 415 may determine the route based on the region information. The route determination module 415 may determine the route according to a route determination algorithm or a moving model. Exemplary route determination algorithms may include a rapidly exploring random tree (RRT) algorithm, a breath first search (BFS) algorithm, a Dijkstra algorithm, an A-star algorithm, an LPA-star algorithm, a D-star algorithm, or the like, or a combination thereof. In some embodiments, the moving model may be or include a machine learning model. Exemplary machine learning models may include multiple layer perceptron (MLP) model, a gradient boosting decision tree (GBDT) model, an extreme gradient boosting (XGB) model, a logistic regression model, and a factorization machine (FM) model, or the like, or any combination thereof. In some embodiments, the model may be selected from the group consisting of a multiple layer perceptron (MLP) model, a gradient boosting decision tree (GBDT) model, an extreme gradient boosting (XGB) model, a logistic regression model, and a factorization machine (FM) model.

The driving controlling module 420 may cause the scanning apparatus 110 to move to the target position along the route. The scanning apparatus 110 may be driven to move to the target position along the route by the driving device 115. The driving controlling module 420 may control a moving speed at which the driving device 115 drives the scanning apparatus 110 to move to the target position. In some embodiments, the moving speed of the scanning apparatus 110 may be a constant, such as 5 kilometers/hour (Km/h), 10 Km/h, 15 Km/h, 20 Km/h, etc. In some embodiments, the moving speed of the scanning apparatus 110 may vary according to different situations. In some embodiments, the processing device 120 may determine the moving speed of the scanning apparatus 110 based on the route condition information.

The identification module 425 may identify a target portion of the subject. In some embodiments, the identification module 425 may identify the target portion based on image data obtained from at least one optical device set on the scanning apparatus 110. The at least one optical device that is used to identify the target portion of the subject may also be referred to as first optical device. In some embodiments, the at least one first optical device may include an optical camera, a digital camera, an infrared camera, a video recorder, etc.

When the scanning apparatus 110 is moved to the target position, the at least one first optical device may generate image data of at least one optical sensing area. The identification module 425 may obtained the image data and identify a target portion of the subject based on the image data. In some embodiments, the image data may include one or more images of the at least one optical sensing area.

In some embodiments, the identification module 425 may identify the target portion from the one or more images of the at least one optical sensing area based on an identification algorithm and/or an identification model. The identification algorithm or the identification model may be used to identify the target portion of the subject in an image based on features (e.g., a shape, a size, grey values, an outline, etc.) of the target portion.

The scan controlling module 430 may cause the scanning apparatus 110 to scan the target portion of the subject. In some embodiments, the image data including the target portion may indicate position information of the target portion. The position information may include a position of the target portion relative to a reference point, a reference line, and/or a reference plane, coordinates in a coordinate system (e.g., the coordinate system 170 illustrated in FIG. 1), a positioning direction of the target portion relative to a reference direction, and/or a reference plane, or the like, or any combination thereof.

The scan controlling module 430 may determine a position difference and/or a direction difference between the target portion and the scanning apparatus 110 based on the position information of the target portion. The scan controlling module 430 may cause the scanning apparatus 110 to adjust at least one of a position or a positioning direction (also referred to as posture adjustment) of the scanning apparatus 110 according to the position difference and the direction difference. After the position difference and the direction difference between the target portion and the scanning apparatus 110 is determined, the scanning apparatus 110 may be driven, by the driving device 115, to move by the position difference and/or move by the direction difference.

After the position and/or positioning direction of the scanning apparatus 110 is adjusted, the target portion of the subject may be positioned in the detecting region 113 of the scanning apparatus 110. Then the scan controlling module 430 may cause the scanning apparatus 110 to perform a scan (e.g., an imaging scan) on, for example, a scanning region which includes the target portion of the subject. The scan may be performed according to a scanning protocol.

The charging control module 435 may cause the scanning apparatus 110 to initiate charging at the contactless charging station or the contact charging station. After the scan performed on the subject is complete, the scanning apparatus 110 may be currently vacant. The scanning apparatus 110 may be moved to a charging area for charging the scanning apparatus 110. In some embodiments, the charging area may include a charging station. The charging station may include one or more devices or components (e.g., a power source, a charging port, etc.) for charging. The charging station may be a contactless charging station or a contact charging station. The charging control module 435 may cause the scanning apparatus 110 to initiate charging at the contactless charging station or the contact charging station.

The modules in the processing device 120 may be connected to or communicated with each other via a wired connection or a wireless connection. The wired connection may include a metal cable, an optical cable, a hybrid cable, or the like, or any combination thereof. The wireless connection may include a Local Area Network (LAN), a Wide Area Network (WAN), a Bluetooth, a ZigBee, a Near Field Communication (NFC), or the like, or any combination thereof. Two or more of the modules may be combined into a single module, and any one of the modules may be divided into two or more units. For example, the processing device 120 may include a storage module (not shown) configured to store information and/or data (e.g., region information of the region, sensing data of one or more sensors set on the scanning apparatus 110, scanning data of the subject, images of the subject, etc.) associated with the above-mentioned modules.

FIG. 4B is a block diagram illustrating an exemplary power module of the scanning apparatus according to some embodiments of the present disclosure. The power module 450 of the scanning apparatus 110 may facilitate charging and/or discharging of one or more devices or components of the scanning apparatus 110. As illustrated in FIG. 4B, the power module 450 may include a power storage device 455, a power receiver 460, a processing circuit 465, a charging circuit 470, and a monitoring device 475.

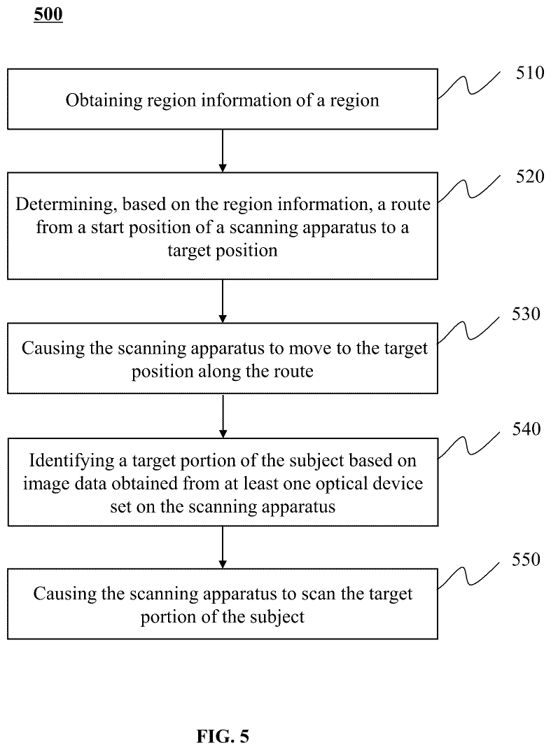

The power storage device 455 may store power and/or provide power to one or more devices or components of the scanning apparatus 110 to perform a scan (e.g., the scan in 550 of the process 500 as illustrated in FIG. 5) and/or drive the scanning apparatus 110 to move to a position in the region (e.g., the target position, the charging area, etc.). The power storage device 455 may be, for example, a battery or a battery assembly. The battery may be a rechargeable battery.

The power receiver 460 may electrically connect to a power transmitter, and receive power from the power transmitter. The power receiver 460 may be electrically connected to the power storage device 455. The power received by the power receiver 460 may be stored into the power storage device 455.

In some embodiments, the power receiver 460 may be a wireless power receiver. The wireless power receiver may include a power receiving coil and a receiving circuit. The power receiving coil may generate an electric current based on the magnetic field induced by the power transmitting coil. The receiving circuit may receive and process the electric current generated in the power receiving coil. The wireless power transmitter may transmit electric power from the power source to the scanning apparatus 110 (e.g., a wireless power receiver of the scanning apparatus 110) wirelessly. The wireless power receiver may be operably connected to the wireless power transmitter at the contactless charging station wirelessly to facilitate the wireless charging of the power storage device 455 of the scanning apparatus 110 when the scanning apparatus 110 is located in the charging area.

The processing circuit 465 may process the electric power received by the power receiver. The processing circuit 465 may process the electric power received by the power receiver 460 by performing a processing operation. Exemplary processing operations may include a rectifying operation, a filtering operation, or the like, or a combination thereof.

In some embodiments, the processing circuit 465 may be or include a rectifier and filter circuit. The rectifying operation and/or the filtering operation may be performed by the rectifier and filter circuit. The rectifier and filter circuit may rectify and filter the electric power (e.g., electric current) from the power receiver 460. For example, the rectifier and filter circuit may transform the electric current from an alternative current to a stable direct current. In some embodiments, the rectifier and filter circuit may include a transformer sub-circuit, a rectifying sub-circuit, a filtering sub-circuit, etc. The transformer sub-circuit may include, for example, a primary winding, a secondary winding, and an iron core. Exemplary rectifying sub-circuits may include a half-wave rectifying sub-circuit, a full-wave rectifying sub-circuit, a bridge rectifying sub-circuit, a voltage multiplier rectifying sub-circuit, etc. Exemplary filtering sub-circuits may include a capacitor filtering sub-circuit, an inductance filtering sub-circuit, an RC filtering sub-circuit, an LC filtering sub-circuit, etc.

The charging circuit 470 may charge the power storage device 455 with the processed electric power. The charging circuit 470 may charge the power storage device 455 with the processed electric power. In some embodiments, the charging circuit 470 may form a low and constant electric current, and charge the power storage device 455 with the low and constant electric current. In some embodiments, the charging circuit 470 may include a plurality of electronic elements. A total voltage and a total resistance of the plurality of electronic elements may be constant, such that the charging circuit 470 may form the low and constant electric current for charging the power storage device 455.

The monitoring device 475 may determine whether the power storage device is in a normal status. The monitoring device 475 may determine a status of the power storage device 455 during the charging of the scanning apparatus 110. An abnormal status of the power storage device 455 may bring about safety hazards for the scanning apparatus 110. In some embodiments, the status of the power storage device may be indicated by at least one of a voltage, a current, or a temperature of the power storage device 455.

In some embodiments, the monitoring device 475 may obtain values of one or more parameters of the power storage device 455. The monitoring device 475 may process or analyze the values of the one or more parameters so as to determine the status of the power storage device 455.

If it is determined that the power storage device is in an abnormal status, the monitoring device 475 may disconnect the current path from the power source to the power storage device 455 immediately to avoid further damages to the power storage device 455. The monitoring device 475 may also generate failure information of the power storage device 455. If it is determined that the power storage device 455 is in a normal status, the monitoring device 475 may control the charging circuit 470 to continue the charging of the power storage device 455 until the power storage device 455 is fully charged.

FIG. 5 is a flowchart illustrating an exemplary process for causing a scanning apparatus to scan a target portion of a subject automatically according to some embodiments of the present disclosure. In some embodiments, the process 500 may be executed by the automated scanning system 100. For example, the process 500 may be implemented as a set of instructions (e.g., an application) stored in a storage device (e.g., the storage device 130). The modules described in FIGS. 4A and 4B and/or the processor 210 may execute the set of instructions and may accordingly be directed to perform the process 500. The operations of the illustrated process presented below are intended to be illustrative. In some embodiments, the process 500 may be accomplished with one or more additional operations not described and/or without one or more of the operations discussed. Additionally, the order of the operations of process 500 illustrated in FIG. 5 and described below is not intended to be limiting.

In 510, the processing device 120 (e.g., the processor 210 or the obtaining module 410) may obtain region information of a region.

The region may be a geographic area in which the scanning apparatus 110 moves, for example, from a start position to a designated position (e.g., a target position where a subject is located). The region may be within a hospital, a laboratory, a workshop, a classroom, etc. For instance, the region may include one or more rooms (e.g., an intensive care unit (ICU), an inpatient room, an outpatient room, etc.) within a hospital or a medical office building.

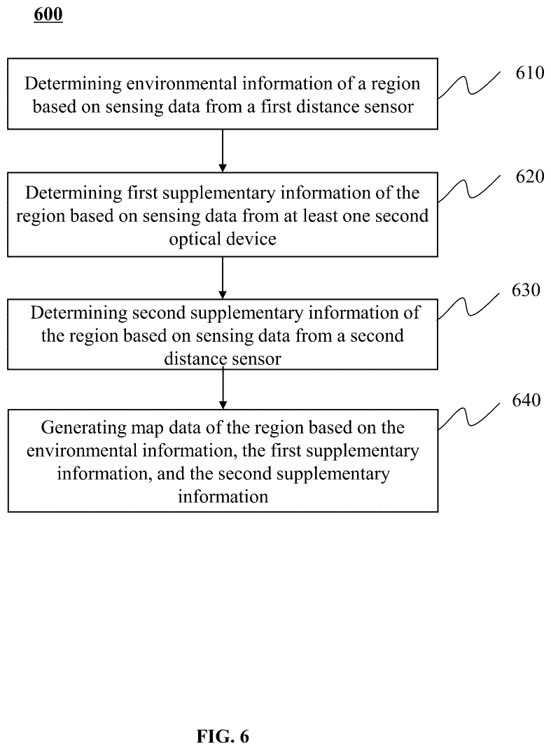

As used herein, the region information refers to information regarding objects and routes in the region. Objects in the region may include, for example, a person, an animal, a table, a door, a wall, a plant, equipment, furniture, a fixture, etc. In some embodiments, the region information may include map data of the region. The map data of the region may include, for example, one or more digital maps of the region. In some embodiments, the map data of the region may be stored in a storage device (e.g., the storage device 130, the storage 220, the storage 309, a cloud storage, etc.). The processing device 120 may retrieve the map data of the region from the storage device. In some embodiments, the processing device 120 may obtain sensing data from one or more sensors set on the scanning apparatus 110 and/or set at one or more positions in the region. The one or more sensors may include at least one distance sensor and at least one optical device. The at least one optical device configured to generate the sensing data may also be referred to as second optical device. Exemplary distance sensors may include a light detection and ranging (LIDAR), a radar, an ultrasound distance sensor, etc. Exemplary second optical devices may include an optical camera, a digital camera (also referred to as camera for brevity), an infrared camera, a video recorder etc. For instance, the one or more sensors may include at least one LIDAR, at least one radar, and/or at least one camera. The one or more positions in the region may include a center of the region, corners of the region, a center of each of one or more sub-regions (e.g., one or more rooms in the region) in the region, corners of each of the one or more sub-regions, and/or other positions determined by, e.g., the user, according to default settings of the automated scanning system 100, etc. Merely for illustration, the one or more sensors may include first sensors set on the scanning apparatus 110 and second sensors (e.g., surveillance cameras) set at a corner of each of one or more rooms or hall ways in a hospital. The processing device 120 may generate the map data of the region based on the sensing data obtained from the one or more sensors. Additional descriptions regarding the generation of the map data of the region may be found elsewhere in the present disclosure. See, for example, FIG. 6 and the descriptions thereof.

In some embodiments, the region information may also include route condition information. The route condition information refers to conditions of routes in the region. The routes may be, for example, potential routes that the scanning apparatus 110 moves in the region. The routes may be determined based on the map data of the region. The route condition information may include, for example, a route width, a route length, movement statuses of moving objects, a count of the moving objects, etc., of each route in the region. The moving objects may be, for example, a doctor, a patient, another scanning apparatus, a wheelchair moving in the region, etc. In some embodiments, the route condition information may be determined based on the map data and/or sensing data from the at least one second optical device set on the scanning apparatus.

In some embodiments, the route condition information may be updated in-real time or intermittently (e.g., periodically or aperiodically). For example, movement statuses of moving objects may vary at different time points. The route condition information, or a portion thereof, may be updated in-real time when the scanning apparatus 110 is driven, e.g., by the driving device 115, to move to a position in the region.

In some embodiments, positions of the moving objects in the region may change at different time points or in a time period. The processing device 120 may identify the moving objects in the region based on the sensing data from the at least one second optical device. The movement status of a moving object may be determined in real-time or intermittently (e.g., periodically or aperiodically). The movement status of a moving object may include, for example, a moving speed, a moving direction, a moving trajectory, acceleration, etc., of the moving object. The processing device 120 may determine or update the route condition information based on the movement statuses of the moving objects. For example, the processing device 120 may determine whether one or more of the moving objects appear on a route of the scanning apparatus 110 when the scanning apparatus 110 is driven to move to a position.

In 520, the processing device 120 (e.g., the processor 210 or the route determination module 415) may determine, based on the region information, a route from a start position of a scanning apparatus to a target position.

The subject may include a biological subject and/or a non-biological subject. The biological subject may be a human being, an animal, a plant, or a specific portion, organ, and/or tissue thereof. For example, the subject may include the head, the neck, the thorax, the heart, the stomach, a blood vessel, soft tissue, a tumor, a nodule, or the like, or any combination thereof, of a patient. In some embodiments, the subject may be a man-made composition of organic and/or inorganic matters that are with or without life. The term “subject” and “object” are used interchangeably in the present disclosure. In some embodiments, the subject may be a patient.

In some embodiments, the start position of the scanning apparatus 110 may be determined by a positioning device (e.g., a global positioning system (GPS), a ZigBee positioning device, etc.) set on or in connection with the scanning apparatus 110. In some embodiments, the start position of the scanning apparatus 110 may be set by a user, for example, on a digital map of the region, via an interface set on the scanning apparatus 110 or the I/O 230.

In some embodiments, the target position may be set by the processing device 120 or a terminal device 140. For example, the target position may be set by a doctor via an interface of the terminal device 140. In some embodiments, the target position may be set by the user, for example, on the digital map of the region, via the interface set on the scanning apparatus 110 or the I/O 230. The target position may be, e.g., an intensive care unit (ICU), an inpatient room, an outpatient room, a laboratory, a classroom, a position where an accident occurs, etc.

In some embodiments, a coordinate system (e.g., the coordinate system 170) may be merged into the map data of the region. The start position of the scanning apparatus and the target position where the subject is located may be represented by different coordinates with reference to the coordinate system.

The processing device 120 may determine, based on the region information, the route from the start position of the scanning apparatus 110 to the target position according to a route determination algorithm or a moving model. Exemplary route determination algorithms may include a rapidly exploring random tree (RRT) algorithm, a breath first search (BFS) algorithm, a Dijkstra algorithm, an A-star algorithm, an LPA-star algorithm, a D-star algorithm, or the like, or a combination thereof.

In some embodiments, the moving model may be or include a machine learning model. Exemplary machine learning models may include multiple layer perceptron (MLP) model, a gradient boosting decision tree (GBDT) model, an extreme gradient boosting (XGB) model, a logistic regression model, and a factorization machine (FM) model, or the like, or any combination thereof. In some embodiments, the model may be selected from the group consisting of a multiple layer perceptron (MLP) model, a gradient boosting decision tree (GBDT) model, an extreme gradient boosting (XGB) model, a logistic regression model, and a factorization machine (FM) model.

The moving model may be trained by inputting a plurality of sample routes and corresponding region information of the region. In some embodiments, the plurality of sample routes may be selected by a user (e.g., a technician, a doctor) at different circumstances. Each of the sample routes may be an optimal route (e.g., a route with a shortest distance and/or a shortest time) from the start position of the scanning apparatus 110 to the target position. The different circumstances may include relevant information of the selected route, such as specific positions or sub-regions in the region, the day (e.g., Monday, Thursday, Sunday), the time period (e.g., 8 A.M.-12 P.M., 2 P.M.-4 P.M., 8 P.M.-10 P.M., etc.). For example, during 8 A.M.-12 P.M. on Monday, a hallway passing through an entrance of a specific outpatient room of a hospital may be crowded according to prior region information of the hospital. A sample route at that circumstance (i.e., during 8 A.M.-12 P.M. on Monday in the hospital) may avoid the gateway. A trained moving model may be generated after the moving model is trained based on the plurality of sample routes and corresponding region information of the region. The processing device 120 may determine the route from the start position of the scanning apparatus 110 to the target position by inputting the region information, the start position, and the target position into the trained moving model.

In some embodiments, the region may be a three-dimensional region. The three-dimensional region may include steps or stairs. In some embodiments, the processing device 120 may identify steps or stairs in the region based on the region information of the region. The route from the start position of the scanning apparatus 110 to the target position may avoid the steps or stairs. In this way, the safety and moving stability of the scanning apparatus 110 may be improved.

In some embodiments, the processing device 120 may obtain appointment/arrangement information regarding an diagnosis and/or treatment of each of one or more patients or work (repairing of a door, an elevator, etc.) of each of one or more staff members or equipment located in the region. The appointment/arrangement information may include a specific time period, a specific location, a status (e.g., over due, on time, ahead of schedule, etc.) of the appointment/arrangement of the diagnosis and/or treatment or work. The route from the start position of the scanning apparatus 110 to the target position may be determined based at least in part on the appointment/arrangement information. Merely for illustration, the processing device 120 may obtain appointment information of repairing of a door in the region during 8 A.M.-10 A.M. A route from the specific position of the scanning apparatus 110 to the target position that passes by the door during 8 A.M.-10 A.M. in the region may be avoided.

In 530, the processing device 120 (e.g., the processor 210 or the driving controlling module 420) may cause the scanning apparatus to move to the target position along the route.

The scanning apparatus 110 may be driven to move to the target position along the route by the driving device 115. In some embodiments, the driving device 115 may include a driver. In some embodiments, the driver may be or include a motor. The motor may include a low speed motor (e.g., a gear motor, a claw pole synchronous motor), a high speed motor, a constant speed motor, a variable speed motor (e.g., an electromagnetic variable-speed motor, a speed-switched reluctance motor, a DC speed motor), a linear motor, or the like, or any combination thereof.

The driving device 115 may drive the scanning apparatus 110 to move to the target position at a moving speed. In some embodiments, the moving speed of the scanning apparatus 110 may be a constant, such as 5 kilometers/hour (Km/h), 10 Km/h, 15 Km/h, 20 Km/h, etc. In some embodiments, the moving speed of the scanning apparatus 110 may vary according to different situations. In some embodiments, the processing device 120 may determine the moving speed of the scanning apparatus 110 based on the route condition information. For example, if a moving object (e.g., a doctor) appears in front of the scanning apparatus 110, the processing device 120 may slow down scanning apparatus 110 (e.g., the moving speed of the scanning apparatus 110 may be decreased to zero). In the meanwhile, a voice message may be broadcasted to the moving object to prompt the moving object to keep away from the scanning apparatus 110 by a loudspeaker set on the scanning apparatus 110. As another example, if there are a plurality of moving objects moving in the vicinity of the route, the route may be determined as a crowded route, and the moving speed of the scanning apparatus 110 may be limited to a value below a threshold (e.g., 2 Km/h, 5 Km/h, 8 Km/h, etc.). As a further example, if there are no moving objects on the route, the route may be determined as a clear route, and the moving speed of the scanning apparatus 110 may be set to a maximum value. The processing device 120 may cause the scanning apparatus 110 to move to the target position along the route at the determined moving speed.

In some embodiments, multiple moving modes may be provided. Merely by way of example, the multiple moving modes may include an urgent mode, a routine mode, a safe mode, etc. The processing device 120 may select a moving mode from the multiple moving modes before the route from the start position to the target position is determined. The processing device 120 may determine the route from the start position to the target position and cause the scanning apparatus 110 to move to the target position along the route in accordance with the selected moving mode. The processing device 120 may select the moving mode from the multiple moving modes according to, for example, the region information, user instructions, actual needs, or the like, or a combination thereof. For example, the processing device 120 may obtain user instructions regarding the moving mode from a user (e.g., a doctor), and select the moving mode according to the user instructions.

In some embodiments, the automated scanning system 100 may communicate with the user, e.g. by sending to and/or receiving from the user voice messages, to facilitate the selection of the moving mode. Merely for illustration, the automated scanning system 100 may send a voice message to the user to inquire a current situation (e.g., whether there is a need for an emergency treatment or a routine scan, potential routes in the region is crowded or not, etc.). For example, if the user determines that there is a need for an emergency treatment, the user may send a first instruction corresponding to the emergency treatment to the automated scanning system 100 via a voice message. After the automated scanning system 100 receives the first instruction, the processing device 120 may select the urgent mode under which the route from the start position to the target position may be determined. Under the urgent mode, the one or more sensors (e.g., the at least one distance sensor, the at least one second optical device, etc.) may be used to determine and/or update the region information (e.g., the route condition information), and a route with a shortest distance and/or a shortest time may be determined based on the route condition information. When the scanning apparatus 110 moves to the target position along the route, a voice message and/or an alarm may be broadcasted by a loudspeaker set on the scanning apparatus 110 to prompt moving objects on the route to keep away from the scanning apparatus 110.

As another example, if the user determines that there is a need for a routine diagnosis and/or treatment, the user may send a second instruction corresponding to the routine diagnosis and/or treatment to the automated scanning system 100 via a voice message. After the automated scanning system 100 receives the second instruction, the processing device 120 may select the routine mode under which the route from the start position to the target position may be determined. Under the routine mode, the one or more sensors (e.g., the at least one distance sensor, the at least one second optical device, etc., as described in FIG. 6) may be used to determine and/or update the region information (e.g., the route condition information), and a relatively clear route (e.g., a route with least moving objects) may be determined based on the route condition information. When the scanning apparatus 110 moves to the target position along the route, the scanning apparatus 110 (e.g., a portion of the one or more sensors) may detect moving objects on the route at a relatively low frequency and a moving speed of the scanning apparatus 110 may be set to a relatively higher value (e.g., 20 Km/h).

As a further example, if the user determines that the region is crowded, the user may send a third instruction corresponding to the crowded condition to the automated scanning system 100 via a voice message. After the automated scanning system 100 receives the third instruction, the processing device 120 may select the safe mode under which the route from the start position to the target position may be determined. Under the safe mode, the one or more sensors (e.g., the at least one distance sensor, the at least one second optical device, etc.) may be used to determine and/or update the region information (e.g., the route condition information), and a route having a relatively large width may be determined based on the route condition information. When the scanning apparatus 110 moves to the target position along the route, the scanning apparatus 110 may detect moving objects on the route at a relatively high frequency and a moving speed of the scanning apparatus 110 may be limited to a value below a threshold (e.g., 2 Km/h, 5 Km/h, 8 Km/h, etc.).

In some embodiments, the automated scanning system 100 may communicate with the user intermittently (e.g., periodically or aperiodically) to inquire the current situation. If the current situation changes (e.g., an emergency occurs), the moving mode may be switched to a corresponding mode (e.g., the urgent mode) immediately according to the current situation. It should be noted that the selection of the moving mode from the multiple moving modes according to user instructions is merely provided for illustration purposes and not intended to be limiting. In some embodiments, the selection of the moving mode may also be implemented by the automated scanning system 100 automatically using the one or more sensors and/or various information obtained from a control center of the region (e.g., videos from one or more surveillance cameras set in the region, an emergency reception record, etc.), the Internet, a local area network, a storage device, etc.

In 540, the processing device 120 (e.g., the processor 210 or the identification module 425) may identify a target portion of the subject based on image data obtained from at least one optical device set on the scanning apparatus.

The at least one optical device that is used to identify the target portion of the subject may also be referred to as first optical device. The at least one first optical device may be configured to generate image data including a representation of the subject. In some embodiments, the at least one first optical device may include an optical camera, a digital camera, an infrared camera, a video recorder, etc. The at least one first optical device may be set on the scanning apparatus 110 (e.g., on the gantry 111 of the scanning apparatus 110). In some embodiments, the at least one first optical device and the at least one second optical device may share one or more optical devices.

The at least one first optical device may face one or more specific directions (also referred to as facing directions). In some embodiments, the at least one first optical device may be arranged around the scanning apparatus 110 (e.g., a periphery of the gantry 111 of the scanning apparatus 110) in the X-Y plane of the coordinate system 170 as illustrated in FIG. 1. Each of the at least one first optical device may generate sensing data (e.g., image data) of a corresponding optical sensing area in the region. The optical sensing area may correspond to a field of view (FOV) of the first optical device. The optical sensing area may be a fan-shaped area symmetrical about a facing direction of the first optical device. The fan-shaped area may have a specific angle (e.g., 60 degrees, 90 degrees, 120 degrees, 150 degrees, etc.).

Merely for illustration, the at least one first optical device may include four cameras. The four cameras may be arranged on four sides of the gantry 111 of the scanning apparatus 110. Facing directions of the four cameras may include a positive Y direction, a negative Y direction, a positive X direction, and a negative X direction. The cameras having facing directions in the positive Y direction and the negative Y direction may be arranged on the gantry 111 above the detecting region 113. The four cameras may generate image data of four optical sensing areas. Each of the four optical sensing areas may be a fan-shaped area having an angle of 120 degrees.

When the scanning apparatus 110 is moved to the target position, the at least one first optical device may generate image data of at least one optical sensing area. The processing device 120 may obtained the image data and identify a target portion of the subject based on the image data. In some embodiments, the image data may include one or more images of the at least one optical sensing area.

The target portion may be, for example, a specific organ, specific tissue, etc., of the subject. Merely by way of example, the target portion may be the head, the neck, etc., of a patient. The target portion may be set by a user, according to default settings of the automated scanning system 100, etc. For instance, the target portion may be obtained from the interface set on the scanning apparatus 110 (e.g., the gantry 111 of the scanning apparatus 110), through which a user may input the target portion of the subject (e.g., the head, the arm, the lung, etc., of the patient). In some embodiments, the target portion may be the head of a patient.

In some embodiments, the processing device 120 may identify the target portion from the one or more images of the at least one optical sensing area based on an identification algorithm and/or an identification model. The identification algorithm or the identification model may be used to identify the target portion of the subject in an image based on features (e.g., a shape, a size, grey values, an outline, etc.) of the target portion. Exemplary identification algorithms may include a scale-invariant feature transform (SIFT) algorithm, a speed up robust feature (SURF) algorithm, a features from accelerated segment test (FAST) algorithm, a binary robust independent elementary features (BRIEF) algorithm, an oriented FAST and rotated BRIEF (ORB) algorithm, or the like, or a combination thereof.

Exemplary identification models may include a deep belief network (DBN), a Stacked Auto-Encoders (SAE), a logistic regression (LR) model, a support vector machine (SVM) model, a decision tree model, a Naive Bayesian Model, a random forest model, or a Restricted Boltzmann Machine (RBM), a Gradient Boosting Decision Tree (GBDT) model, a Lambda MART model, an adaptive boosting model, a recurrent neural network (RNN) model, a convolutional network model, a hidden Markov model, a perceptron neural network model, a Hopfield network model, or the like, or any combination thereof. In some embodiments, the identification model may be a machine learning model. In some embodiments, the identification model may be a model trained based on a plurality of sample images including target portions of multiple subjects. Merely for illustration, a plurality of sample images, each of which includes the head of a patient, may be obtained. Features (e.g., a shape, a size, an outline, etc.) of the head of the patient in each of the plurality of sample images may be extracted and used to train the identification model.

In some cases, if the processing device 120 does not identify the target portion of the subject based on the image data obtained from the at least one first optical device, the automated scanning system 100 may initiate a searching process to capture and identify the target portion. During this process, a position or a positioning direction of the scanning apparatus 110 and/or a facing direction of each of one or more of the at least one first optical device may be adjusted in a predetermined manner until the target portion of the subject is captured and identified. In some embodiments, a direction along an axis of the detecting region 113 of the scanning apparatus 110 (e.g., the negative Y direction of the coordinate system 170 as illustration in FIG. 1) may be defined as the positioning direction of the scanning apparatus 110.

For example, in the case that the processing device 120 does not identify the target portion of the subject, the scanning apparatus 110 may rotate by a first angle. Merely by way of example, the positioning direction of the scanning apparatus 110 may turn by the first angle in the X-Y plane of a coordinate system (e.g., the coordinate system 170 as illustrated in FIG. 1). The first angle may be, for example, 15 degrees, 30 degrees, 45 degrees, 60 degrees, 90 degrees, 135 degrees, 180 degrees, 270 degrees, etc. In some embodiments, the first angle may be set by a user, according to default settings of the automated scanning system 100, etc. In some embodiments, the first angle may be determined such that a total FOV of the at least one first optical device may cover a range of 360 degrees around the scanning apparatus 110. The total FOV refers to a sum of the FOV of each of the at least one first optical device.

As another example, the scanning apparatus 110 may be driven to move along a preset trajectory or in a preset direction over a certain distance. The certain distance may be, for example, 10 centimeters, 20 centimeters, 30 centimeters, 50 centimeters, 1 meter, 1.5 meters, etc. The preset trajectory may be, for example, an “S” shape trajectory, a “V” shape trajectory, or any suitable trajectory specified by a user, according to default settings of the automated scanning system 100, etc. The direction may be, for example, the X direction, the Y direction, the Z direction, etc., of the coordinate system 170.

As a further example, each of one or more of the at least one first optical device may rotate by a second angle. Merely by way of example, a facing direction of a first optical device may turn by the second angle in the X-Y plane and/or Y-Z plane of a coordinate system (e.g., the coordinate system 170 as illustrated in FIG. 1). The second angle may be, for example, 5 degrees, 10 degrees, 15 degrees, 30 degrees, 45 degrees, 60 degrees, 75 degrees, 90 degrees, 120 degrees, 150 degrees, etc. In some embodiments, the second angle may be set by a user, according to default settings of the automated scanning system 100, etc. In some embodiments, the second angle may be determined such that the FOV of the at least one first optical device may cover a range of 360 degrees around the scanning apparatus 110.

In some embodiments, the at least one first optical device may generate image data in real-time or intermittently (e.g., periodically or aperiodically) during this process. The processing device 120 may analyze the image data to determine whether the target portion of the subject is identified immediately after the processing device 120 obtains the image data. The searching process may terminate until the target portion of the subject is captured and identified based on the image data obtained from the at least one first optical device.

In 550, the processing device 120 (e.g., the processor 210 or the scan controlling module 430) may cause the scanning apparatus to scan the target portion of the subject.

In some embodiments, the image data including the target portion may indicate position information of the target portion. The position information may include a position of the target portion relative to a reference point, a reference line, and/or a reference plane, coordinates in a coordinate system (e.g., the coordinate system 170 illustrated in FIG. 1), a positioning direction of the target portion relative to a reference direction, and/or a reference plane, or the like, or any combination thereof. As used herein, the positioning direction of the target portion refers to a direction of the target portion in which the target position is placed in the detecting region 113 of the scanning apparatus 110 for scanning. For instance, as for the head of a patient, the positioning direction may be from the chin of the patient to the top of the head of the patient. The reference point, the reference line, the reference direction, and/or the reference plane may be set by a user, according to default settings of the automated scanning system 100, etc. For instance, the reference point may be an origin of a coordinate system (e.g., the coordinate system 170 illustrated in FIG. 1). The reference line or reference direction may an axis (e.g., the X axis, the Y axis, or the Z axis) of the coordinate system. The reference plane may be a plane (e.g., the X-Y plane, the Y-Z plane, or the X-Z plane) of the coordinate system. In some embodiments, the image data including the target portion may also indicate other information of the target portion of the subject, such as a size, an outline, etc., of the target portion.

The processing device 120 may determine a position difference and/or a direction difference between the target portion and the scanning apparatus 110 based on the position information of the target portion. In some embodiments, the processing device 120 may obtain the position and the positioning direction of the scanning apparatus 110. The processing device 120 may determine a difference between the position of the target portion and the position of the scanning apparatus 110. The difference between the position of the target portion and the position of the scanning apparatus 110 may be determined as the position difference between the target portion and the scanning apparatus 110. Also, the processing device 120 may determine a difference between the positioning direction of the target portion and the positioning direction of the scanning apparatus 110. The difference between the positioning direction of the target portion and the positioning direction of the scanning apparatus 110 may be determined as the direction difference between the target portion and the scanning apparatus 110. In some embodiments, the position difference may be represented by a distance, and the direction difference may be represented by an angle.

The processing device 120 may cause the scanning apparatus 110 to adjust at least one of a position or a positioning direction (also referred to as posture adjustment) of the scanning apparatus 110 according to the position difference and the direction difference. After the position difference and the direction difference between the target portion and the scanning apparatus 110 is determined, the scanning apparatus 110 may be driven, by the driving device 115, to move by the position difference and/or move by the direction difference.

After the position and/or positioning direction of the scanning apparatus 110 is adjusted, the target portion of the subject may be positioned in the detecting region 113 of the scanning apparatus 110. Then the scanning apparatus 110 may perform a scan (e.g., an imaging scan) on, for example, a scanning region which includes the target portion of the subject. The scan may be performed according to a scanning protocol. The scanning protocol may include parameters (e.g., a scanning voltage, a scanning current) of the scanning apparatus 110, a scanning mode (e.g., spiral scanning, axial scanning) of the scanning apparatus 110, a size of the scanning region, position information of the scanning region, information regarding image contrast and/or ratio, or the like, or any combination thereof. Further, one or more images of the target region of the subject may be generated based on scanning data generated in the scan performed by the scanning apparatus 110.

It should be noted that the above description of the process 500 is provided for the purposes of illustration, not intended to limit the scope of the present disclosure. For persons having ordinary skills in the art, multiple variations and modifications may be reduced to practice in the light of the present disclosure. However, these variations and modifications fall in the scope of the present disclosure.

For example, the processing device 120 may obtain, e.g., via the I/O 230, voice data regarding a posture adjustment of the scanning apparatus 110 from a user, and cause the scanning apparatus 110 to adjust at least one of a position or a positioning direction of the scanning apparatus 110 according to the voice data. The voice data may be or include instructions regarding posture adjustment such as “left turn,” “right turn,” “move forward,” “stop,” “rotate clockwise,” “20 centimeters forward,” etc. After the processing device 120 obtains the voice data, the processing device 120 may transform the voice data into text. The voice data may be transformed into text according to a voice recognition algorithm. Exemplary voice recognition algorithms may include Hidden Markov Models (HMMs), Dynamic Time Warping (DTW)-Based Speech Recognition, Neural Networks, Deep Feedforward and Recurrent Neural Networks (DNN), End-to-End Automatic Speech Recognition (ASR), or the like, or any combination thereof. In some embodiments, acoustic modeling and/or language modeling may be used in the voice recognition algorithm. In some embodiments, the text may include a speech including one or more instructions regarding posture adjustment. The processing device 120 may extract the instructions regarding a posture adjustment from text of the speech. The processing device 120 may cause the scanning apparatus 110 to adjust at least one of a position or a positioning direction of the scanning apparatus 110 according to the instructions regarding posture adjustment.

As another example, in some cases (e.g., an emergency, a failure of the identification of the target portion of the subject), the posture adjustment of the scanning apparatus 110 may be accomplished by a user (e.g., a doctor). The user may adjust at least one of a position or a positioning direction of the scanning apparatus 110 manually.

As a further example, the scanning apparatus 110 may further include a plurality of positioning sensors configured to determine a position of the subject relative to the a scanning table that supports the subject. In some embodiments, the position of the subject relative to the scanning table may be used for posture adjustment of the scanning apparatus 110. For example, the position information of the target portion may be determined based at least partially on the position of the subject relative to the scanning table. In some embodiments, the position of the subject relative to the scanning table may be used in the scan performed in 550. In some embodiments, the plurality of positioning sensors may be or include pressure sensors. Exemplary pressure sensors may include a piezoelectric sensor, a piezoresistive sensor, a ceramic pressure sensor, a diffused silicon pressure sensor, or the like, or a combination thereof. The plurality of positioning sensors may be set at different positions on the scanning table. The different positions may correspond to various portions of the subject. For example, the different positions may correspond to various body parts of a patient. Merely for illustration, the scanning apparatus 110 may include five pressure sensors. The five pressure sensors may be set at specific positions on the scanning table corresponding to the head, the hands, and the feet of the patient, respectively.

If each of the various portions of the subject is placed at a corresponding position where a pressure sensor is set, the pressure sensor may generate a signal indicating that the portion of the subject is placed at the corresponding position. The position of the subject relative to the scanning table may be determined. If one or more of the various portions of the subject are not placed at corresponding positions, the processing device 120 may generate, e,g., via the I/O 230, a voice message to prompt a user (e.g., a doctor, a patient, etc.) to adjust the position of the subject relative to the scanning table.

In some embodiments, the different positions that correspond to the various portions of the subject may change dynamically according to a size of the subject (e.g., a height of a patient). For example, as for a child, a distance between each pair of the positions may be relatively shorter. As for an adult, the distance may be relatively longer. In some embodiments, the processing device 120 may obtain a size of the subject (e.g., via the I/O 230, image data from the at least one first optical device, etc.). The processing device 120 may determine the different positions that correspond to the various portions of the subject based on the size of the subject. Merely for illustration, the processing device 120 may obtain a height of a patient on the scanning table by, e.g., retrieving basic information of the patient from a database. The processing device 120 may determine, e.g., via a big data analysis or historical data, five positions that correspond to the head, the hands, and the feet of the patient, respectively, based on the height of the patient.

As still a further example, operations in 530 and 540 of the process 500 may be performed simultaneously. During the process that the scanning apparatus 110 moves to the target position, the processing device 120 may identify the target portion of the subject continuously based on image data obtained from the at least one first optical device.

As still a further example, the scanning apparatus 110 may be blocked (e.g., by a moving object) or stopped (e.g., by the processing device 120) at a position on the route from the start position to the target position. The position may also be referred to as an intermediate position between the start position and the target position. When it is determined to resume the movement of the scanning apparatus 110 to the target position, the processing device 120 may determine an updated route from the intermediate position between the start position and the target position to the target position. The updated route may be determined based on the region information generated at the time of restarting the scanning apparatus 110. In some embodiments, the determination of the updated route may be similar to or the same as the determination of the route from the start position to the target position, the description of which is not repeated here.

FIG. 6 is a flowchart illustrating an exemplary process for generating map data of a region according to some embodiments of the present disclosure. In some embodiments, the process 600 may be executed by the automated scanning system 100. For example, the process 600 may be implemented as a set of instructions (e.g., an application) stored in a storage device (e.g., the storage device 130). The modules described in FIGS. 4A and 4B and/or the processor 210 may execute the set of instructions and may accordingly be directed to perform the process 600. The operations of the illustrated process presented below are intended to be illustrative. In some embodiments, the process 600 may be accomplished with one or more additional operations not described and/or without one or more of the operations discussed. Additionally, the order of the operations of process 600 illustrated in FIG. 6 and described below is not intended to be limiting. As described above, the region information of the region may include the map data of the region. The map data may be generated according to the operations 610 through 640 of the process 600.

In 610, the processing device 120 (e.g., the processor 210 or the obtaining module 410) may determine environmental information of a region based on sensing data from a first distance sensor.

In some embodiments, the first distance sensor may be a light detection and ranging device (LIDAR). The LIDAR may be set on the scanning apparatus 110. For example, the LIDAR may be set on the gantry 111 of the scanning apparatus 110 facing a moving direction of the scanning apparatus 110. In some embodiments, the LIDAR may include a light transmitter and a light receiver. The light transmitter may transmit light pulses to environment around the scanning apparatus 110 in the region. The light pulses may include pulses of ultraviolet light, visible light, and/or near infrared light. In some embodiments, the light pulses may be pulses of laser. At least a portion of the transmitted light pulses may be reflected by specific objects, such as walls, doors, tables, etc., in the environment in the region. The reflected light pulses may be received by the light receiver.

In some embodiments, point clouds may be generated based on the received light pulses. The point clouds may include a set of points that represent 3D features (e.g., a 3D outline) of the environment in the region. Each of the set of points may include coordinates of the point, a grey value of the point, a depth of the point, etc. The point clouds may be referred to as the sensing data from the first distance sensor. The processing device 120 may determine the environmental information of the region based on the point clouds. The environmental information may include the set of points representing 3D features, grey values, etc., of the environment in the region.

In 620, the processing device 120 (e.g., the processor 210 or the obtaining module 410) may determine first supplementary information of the region based on sensing data from at least one second optical device.

In some embodiments, each of the at least one second optical device may be or include a camera. The at least one second optical device may be set on the scanning apparatus 110. For example, the at least one second optical device may be set around the scanning apparatus (e.g., a periphery of the gantry of the scanning apparatus 110). Each of the at least one second optical device may generate image data of a corresponding optical sensing area in the region. The optical sensing area may correspond to a field of view (FOV) of the second optical device. In some embodiments, the optical sensing area may be a fan-shaped area symmetrical about a facing direction of the second optical device. The fan-shaped area may have a specific angle (e.g., 60 degrees, 90 degrees, 120 degrees, 150 degrees, etc.). The image data of the optical sensing area corresponding to each of the at least one second optical device may be referred to as sending data from the at least one second optical device.

The LIDAR may have blind spots or areas. For example, the light pulses generated by the LIDAR may transmit to the environment in a specific angle range. The specific angle range may be, for example, 90 degrees, 120 degrees, 150 degrees, etc. The LIDAR may not detect objects in one or more spots or areas out of the specific angle range in the region effectively. The one or more spots or areas may be the blind spots or areas of the LIDAR. In some embodiments, a count and/or position of the at least one second optical device may be determined such that the at least one second optical devices may generate image data including at least the blind spots or areas of the LIDAR. Thus, the sensing data from the at least one second optical devices may be determined as supplementary information (also referred to as first supplementary information) for the environmental information determined based on the sensing data from the LIDAR. The first supplementary information may include at least information (e.g., positions, shapes, etc.) regarding the objects in the blind spots or areas of the LIDAR.

In 630, the processing device 120 (e.g., the processor 210 or the obtaining module 410) may determine second supplementary information of the region based on sensing data from a second distance sensor.

In some embodiments, the second distance sensor may be a radar. The radar may also be set on the scanning apparatus 110. For example, the radar may be set on the gantry 111 of the scanning apparatus 110 facing a moving direction of the scanning apparatus 110. In some embodiments, the radar may include a wave transmitter and a wave receiver. The wave transmitter may transmit radar waves to environment around the scanning apparatus 110 in the region. The radar waves may include microwaves, millimeter waves, and/or near ultrasound waves. In some embodiments, the radar waves may be ultrasound waves. Correspondingly, the radar may be an ultrasound radar. At least a portion of the transmitted radar waves may be reflected by various objects in the environment in the region. The reflected radar waves may be received by the wave receiver. In some embodiments, a pre-processing operation may be performed on the received radar waves. Exemplary pre-processing operations may include an analog-to-digital (AD) conversion, a filtering operation, a gain adjusting operation, a denoising operation, etc. The received radar waves and/or the pre-processed radar waves may be referred to as the sensing data from the second distance sensor.

As for the LIDAR, the light pulses may pass through transparent objects (e.g., glass on doors, windows, etc.) easily. In this case, the LIDAR may not detect the transparent objects in the region effectively. The radar may differ from the LIDAR in that at least a portion of the radar waves emitted by the radar may be reflected by the transparent objects in the region. Thus, the sensing data from the radar may be determined as supplementary information (also referred to as second supplementary information) for the environmental information determined based on the sensing data from the LIDAR. The second supplementary information may include at least information (e.g., positions, sizes, etc.) regarding the transparent objects.

In 640, the processing device 120 (e.g., the processor 210 or the obtaining module 410) may generate map data of the region based on the environmental information, the first supplementary information, and the second supplementary information.

After the environmental information, the first supplementary information, and the second supplementary information of the region is determined, the processing device 120 may generate the map data of the region. Merely for illustration purposes, the processing device 120 may generate primary map data based on the environmental information. For instance, the processing device 120 may generate the primary map data based on the point clouds according to a map reconstruction technique. Exemplary map reconstruction techniques may include a hector_slam technique, a gmapping_slam technique, a karto_slam technique, a core_slam technique, etc. Then the processing device 120 may update the primary map data by supplementing the objects in the blind spots or areas of the LIDAR and transparent objects in the region into the primary map data based on the first supplementary information, and the second supplementary information. The updated map data may be referred to as the map data of the region.

It should be noted that the above description of the process 800 is provided for the purposes of illustration, not intended to limit the scope of the present disclosure. For persons having ordinary skills in the art, multiple variations and modifications may be reduced to practice in the light of the present disclosure. For example, the scanning apparatus 110 may include multiple first distance sensors and multiple second distance sensors. The multiple first distance sensors and the multiple second distance sensors may be set around the scanning apparatus 110 (e.g., a periphery of the gantry 111 of the scanning apparatus 110). As another example, the first distance sensor and the second distance may be replaced with a third distance sensor. The third distance sensor may be, for example, a microwave radar. However, these variations and modifications fall in the scope of the present disclosure.

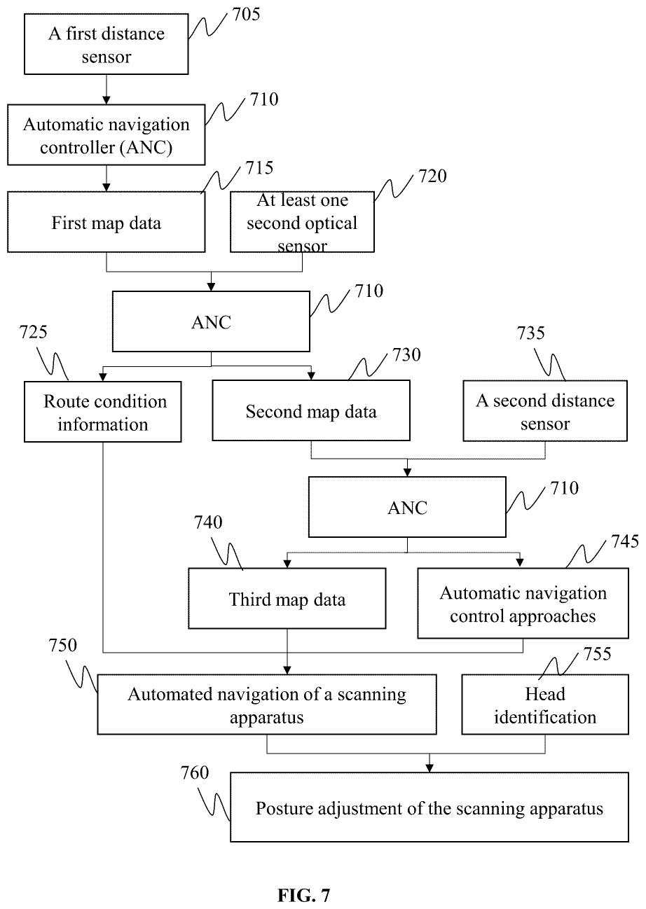

FIG. 7 is a schematic diagram illustrating automated scanning of a target portion of a subject according to some embodiments of the present disclosure. The automated scanning of the target portion of the subject (e.g., the head of a patient) as illustrated in FIG. 7 may be described in connection with FIGS. 5 and 6. A first distance sensor 705 may be set on the scanning apparatus 110. The first distance sensor 705 may be a LIDAR. The LIDAR may transmit light pulses to environment around the scanning apparatus 110 in a region. At least a portion of the transmitted light pulses may be reflected by specific objects, such as walls, doors, tables, etc., in the environment in the region. Point clouds may be generated based on the received light pulses. The point clouds may include a set of points that represent 3D features (e.g., a 3D outline) of the environment in the region. An automatic navigation controller (ANC) 710 may obtain the point clouds, and determine first map data 715 of the region based on the point clouds. The ANC 710 may be an example of the processor 210.

At least one second optical device 720 may also be set on the scanning apparatus 110. Each of the at least one second optical device 720 may be or include a camera. Each of the at least one second optical device 720 may generate image data of a corresponding optical sensing area in the region. The optical sensing area may correspond to a field of view (FOV) of the second optical device 720. The ANC 710 may obtain the image data of the at least one second optical device 720, and determine second map data 730 of the region based on the image data of the at least one second optical device 720 and the first map data 715. In comparison with the first map data 715, the second map data 730 may further include data regarding blind spots or areas of the LIDAR.

In addition, the ANC 710 may further determine route condition information 725 based on the image data of the at least one second optical device 720. The route condition information 725 may include, for example, a route width, a route length, movement statuses of moving objects, a count of the moving objects, etc., of each route in the region. The moving objects may be, for example, a doctor, a patient, another scanning apparatus, etc. In some embodiments, positions and/or moving directions of the moving objects in the region may change. The ANC 710 may identify the moving objects in the region based on the image data of the at least one second optical device 720. The movement statuses of the moving objects may be determined in real-time or intermittently (e.g., periodically or aperiodically). The movement statuses may include, for example, a moving speed, a moving direction, a moving trajectory, etc., of each of the moving objects. The ANC 710 may determine or update the route condition information 725 based on the movement statuses of the moving objects. For example, the ANC 710 may determine whether one or more moving objects appear on a route of the scanning apparatus 110. In a case that a moving object appears on the route of the scanning apparatus 110, a voice message may be broadcasted to the moving object to prompt the moving object to keep away from the scanning apparatus 110 by a loudspeaker set on the scanning apparatus 110.

A second distance sensor 735 may further be set on the scanning apparatus 110. The second distance sensor 735 may be or include a radar. The radar may transmit radar waves to environment around the scanning apparatus 110 in the region. At least a portion of the transmitted radar waves may be reflected by various objects (e.g., transparent objects such as glass on a door, on a window, etc.) in the environment in the region. The ANC 710 may obtain the reflected radar waves, and generate third map data 740 based on the reflected radar waves and the second map data 730. In comparison with the second map data 730, the third map data 740 may further include data regarding transparent objects in the region.

Based on the third map data 740, the route condition information 725, and automatic navigation control approaches 745, an automated navigation 750 of the scanning apparatus 110 from a start position of the scanning apparatus 110 to a target position where the patient is located may be realized. The automatic navigation control approaches 745 may include, for example, a machine learning model, a RRT algorithm, a Dijkstra algorithm, an A-star algorithm, etc.

After the scanning apparatus 110 moves to the target position, head identification 755 may be realized based on image data from at least one first optical device according to an identification algorithm and/or an identification model. The identification algorithm or the identification model may be used to identify the head of the patient in an image based on features (e.g., a shape, a size, grey values, an outline, etc.) of the head. Then a posture adjustment 760 of the scanning apparatus 110 may be conducted such that the head of the patient may be positioned into the detecting region 113 of the scanning apparatus 110. The posture adjustment 760 may be conducted according to a position difference and/or a direction difference between the head of the patient and the scanning apparatus 110.

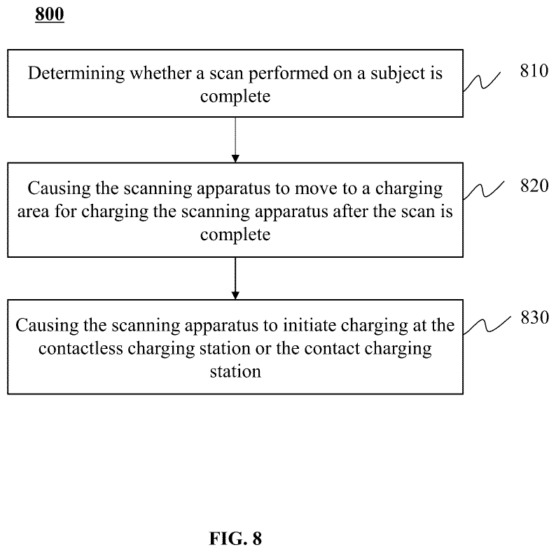

FIG. 8 is a flowchart illustrating an exemplary process for initiating wireless charging of the scanning apparatus according to some embodiments of the present disclosure. In some embodiments, the process 800 may be executed by the automated scanning system 100. For example, the process 800 may be implemented as a set of instructions (e.g., an application) stored in a storage device (e.g., the storage device 130). The modules described in FIGS. 4A and 4B and/or the processor 210 may execute the set of instructions and may accordingly be directed to perform the process 800. The operations of the illustrated process presented below are intended to be illustrative. In some embodiments, the process 800 may be accomplished with one or more additional operations not described and/or without one or more of the operations discussed. Additionally, the order of the operations of process 800 illustrated in FIG. 8 and described below is not intended to be limiting.

In 810, the processing device 120 (e.g., the processor 210 or the scan controlling module 430) may determine whether a scan performed on a subject is complete.

The scan may be, for example, the scan performed on the target portion of the subject as described in 550 of the process 500. In some embodiments, the processing device 120 may determine whether the scanning apparatus 110 is currently vacant. If the scanning apparatus 110 is currently vacant, the processing device 120 may determine that the scan performed on the subject is complete. If the scanning apparatus 110 is performing an operation related to the scan (e.g., emitting radiation rays, obtaining scanning data, reconstructing an image, etc.), the processing device 120 may determine that the scan is not complete.

In 820, processing device 120 (e.g., the processor 210 or the driving controlling module 420) may cause the scanning apparatus to move to a charging area for charging the scanning apparatus after the scan is complete.

The charging area refers to an area where the scanning apparatus 110 is positioned for charging the scanning apparatus 110 (e.g., the power storage device 455 of the scanning apparatus 110). In some embodiments, the charging area may include a charging station. The charging station may include one or more devices or components (e.g., a power source, a charging port, etc.) for charging. The charging station may be a contactless charging station or a contact charging station. The contactless charging station refers to a station for charging the scanning apparatus 110 through an inductive coupling between the scanning apparatus 110 and a power source. The contact charging station refers to a station for charging the scanning apparatus 110 via a physical connection between the scanning apparatus 110 and a power source.

In some embodiments, the charging station may be a contactless charging station. The contactless charging station may include a power source, a wireless power transmitter, etc. The power source may be a battery, an electricity grid, etc. The power source may be electrically connected to the wireless power transmitter. The wireless power transmitter may include a power transmitting coil and a transmitting circuit. The transmitting circuit may control a transmitting parameter (e.g., a current value, a voltage value, etc.) of the electric power in the power transmitting coil. The power transmitting coil may generate a magnetic field based on the electric power in the power transmitting coil. The wireless power transmitter may transmit electric power from the power source to the scanning apparatus 110 (e.g., a wireless power receiver of the scanning apparatus 110) wirelessly.

After the scan is complete, the processing device 120 may obtain a position of the charging area (e.g., in the form of coordinates in the coordinate system 170 as illustrated in FIG. 1) and cause the scanning apparatus 110 to move to the charging area for charging the scanning apparatus 110 at the contactless charging station or the contact charging station. In some embodiments, the charging area may be in the region of which region information may be obtained in 510 of the process 500 as illustrated in FIG. 5. The scanning apparatus 110 may be driven to the charging area by the driving device 115 based on the region information of the region.

In 830, the processing device 120 (e.g., the processor 210 or the charging controlling module 435) may cause the scanning apparatus to initiate charging at the contactless charging station or the contact charging station.

The power module 450 of the scanning apparatus 110 may include the power storage device 455 and the power receiver 460. When the scanning apparatus 110 is located in the charging area, the processing device 120 may cause the scanning apparatus 110 (e.g., the power receiver 460) to initiate charging of the power storage device 455. The power storage device 455 may store power and/or provide power to one or more devices or components of the scanning apparatus 110 to perform a scan (e.g., the scan in 550 of the process 500 as illustrated in FIG. 5) and/or drive the scanning apparatus 110 to move to a position in the region (e.g., the target position, the charging area, etc.). The power storage device 455 may be, for example, a battery or a battery assembly. The battery may be a rechargeable battery. The power storage device 455 may be charged via the power receiver 460, which may be electrically connected to the power storage device 455. The power receiver 460 may electrically connect to a power transmitter, and receive power from the power transmitter. The power received by the power receiver 460 may be stored into the power storage device.

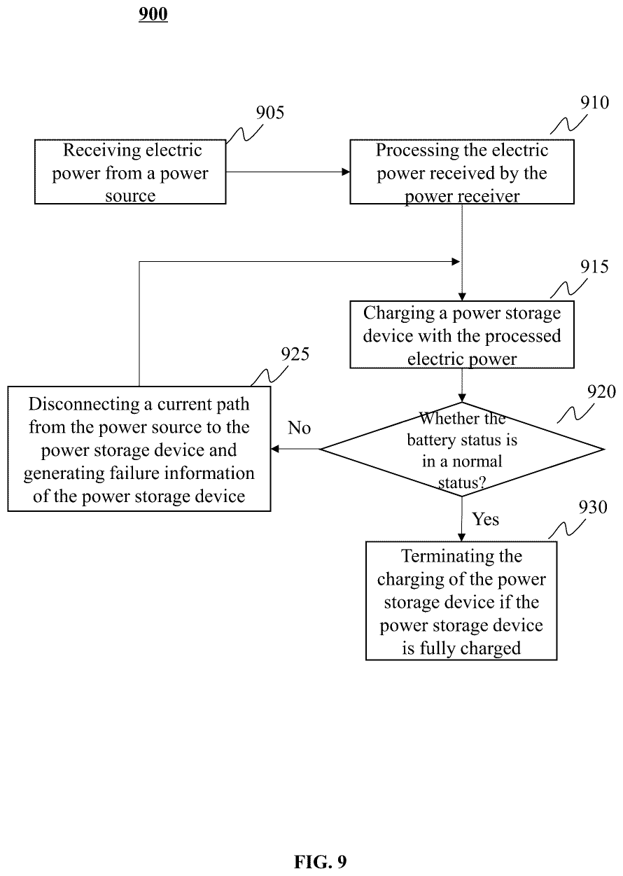

In some embodiments, the power receiver 460 may be a wireless power receiver. The wireless power receiver may include a power receiving coil and a receiving circuit. The power receiving coil may generate an electric current based on the magnetic field induced by the power transmitting coil. The receiving circuit may receive and process the electric current generated in the power receiving coil. The wireless power transmitter may transmit electric power from the power source to the scanning apparatus 110 (e.g., a wireless power receiver of the scanning apparatus 110) wirelessly. The wireless power receiver may be operably connected to the wireless power transmitter at the contactless charging station wirelessly to facilitate the wireless charging of the power storage device 455 of the scanning apparatus 110 when the scanning apparatus 110 is located in the charging area. Further descriptions regarding the wireless charging of the scanning apparatus 110 (e.g., the power storage device 455) may be found elsewhere in the present disclosure. See, for example, FIG. 9 and the descriptions thereof.

FIG. 9 is a schematic diagram illustrating wireless charging of the scanning apparatus according to some embodiments of the present disclosure. In some embodiments, the process 900 may be executed by the automated scanning system 100. For example, the process 900 may be implemented as a set of instructions (e.g., an application) stored in a storage device (e.g., the storage device 130). The modules described in FIG. 4B and/or the processor 210 may execute the set of instructions and may accordingly be directed to perform the process 900. The operations of the illustrated process presented below are intended to be illustrative. In some embodiments, the process 900 may be accomplished with one or more additional operations not described and/or without one or more of the operations discussed. Additionally, the order of the operations of process 900 illustrated in FIG. 9 and described below is not intended to be limiting.

In 905, the power module 450 (e.g., the power receiver 460) may receive electric power from a power source.

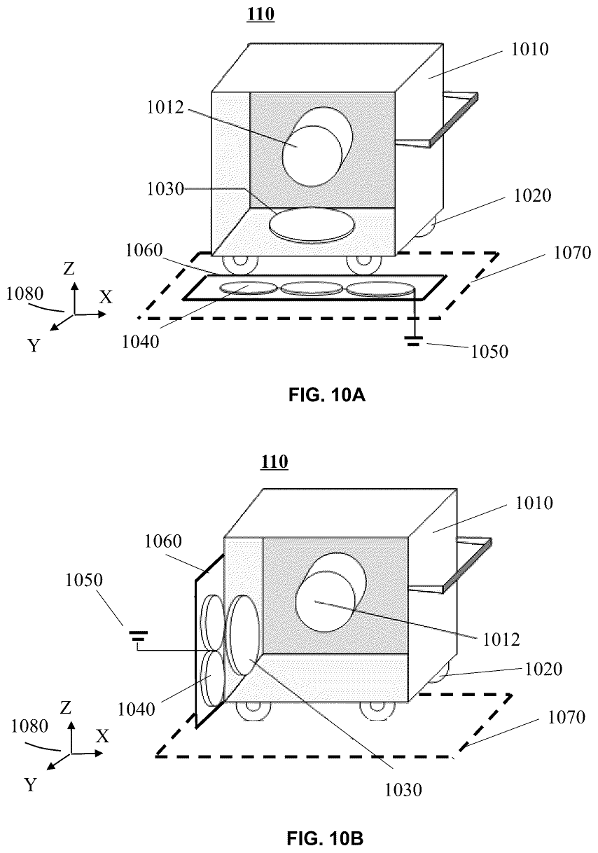

In some embodiments, the power receiver 460 may be a wireless power receiver. When the scanning apparatus 110 is located in a charging area, the power receiver 460 may be operably connected to a wireless power transmitter at a contactless charging station in the charging area. The wireless power transmitter may be electrically connected to a power source (e.g., a battery, an electricity grid, etc.), and transmit electric power from the power source to the power receiver 460. The electric power transmitted to the power receiver 460 may be stored in the power storage device 455 of the scanning apparatus 110. Details regarding structures and arrangements of the wireless power receiver and the wireless power transmitter may be found elsewhere in the present disclosure. See, for example, FIGS. 10A and 10B and the descriptions thereof.

In 910, the power module 450 (e.g., the processing circuit 465) may process the electric power received by the power receiver.

The processing circuit 465 of the power module 450 may process the electric power received by the power receiver 460 by performing a processing operation. Exemplary processing operations may include a rectifying operation, a filtering operation, or the like, or a combination thereof.

In some embodiments, the processing circuit 465 may be or include a rectifier and filter circuit. The rectifying operation and/or the filtering operation may be performed by the rectifier and filter circuit. The rectifier and filter circuit may rectify and filter the electric power (e.g., electric current) from the power receiver 460. For example, the rectifier and filter circuit may transform the electric current from an alternative current to a stable direct current. In some embodiments, the rectifier and filter circuit may include a transformer sub-circuit, a rectifying sub-circuit, a filtering sub-circuit, etc. The transformer sub-circuit may include, for example, a primary winding, a secondary winding, and an iron core. Exemplary rectifying sub-circuits may include a half-wave rectifying sub-circuit, a full-wave rectifying sub-circuit, a bridge rectifying sub-circuit, a voltage multiplier rectifying sub-circuit, etc. Exemplary filtering sub-circuits may include a capacitor filtering sub-circuit, an inductance filtering sub-circuit, an RC filtering sub-circuit, an LC filtering sub-circuit, etc.

In 915, the power module 450 (e.g., the charging circuit 470) may charge the power storage device with the processed electric power.

The charging circuit 470 of the power module 450 may charge the power storage device 455 with the processed electric power obtained in 910. In some embodiments, the charging circuit 470 may form a low and constant electric current, and charge the power storage device 455 with the low and constant electric current. In some embodiments, the charging circuit 470 may include a plurality of electronic elements. A total voltage and a total resistance of the plurality of electronic elements may be constant, such that the charging circuit 470 may form the low and constant electric current for charging the power storage device 455.

In 920, the power module 450 (e.g., the monitoring device 475) may determine whether the power storage device is in a normal status.

The monitoring device 475 may determine a status of the power storage device 455 during the charging of the scanning apparatus 110. An abnormal status of the power storage device 455 may bring about safety hazards for the scanning apparatus 110. Merely for illustration, if a battery cell of the power storage device 455 mounted in the scanning apparatus 110 is in an abnormal status, the power storage device may have a risk of a short circuit, an open circuit, a temperature rise, a change in the electrical resistance, etc. In some embodiments, the status of the power storage device may be indicated by at least one of a voltage, a current, or a temperature of the power storage device 455.

In some embodiments, the monitoring device 475 may obtain values of one or more parameters of the power storage device 455. The one or more parameters may relate to, for example, a voltage value, a current value, and/or the temperature, of the power storage device 455. In some embodiments, the one or more parameters may include one or more voltage related parameters, one or more current related parameters, one or more electrical resistance related parameters, one or more temperature related parameters, etc., of the power storage device 455. A voltage related parameter refers to a parameter relating to a voltage associated with one or more components (e.g., battery cells) of the power storage device 455, such as a voltage of a component (e.g., a battery cell) of the power storage device 455, an average voltage (e.g., an arithmetic average voltage) of one or more components of the power storage device 455, etc. A current related parameter refers to a parameter relating to a current associated with one or more components (e.g., battery cells) of the power storage device 455, such as a current of a component of the power storage device 455, an average current (e.g., an arithmetic average current) of one or more components of the power storage device 455, etc. An electrical resistance related parameter refers to a parameter relating to an electrical resistance associated with one or more components (e.g., battery cells) of the power storage device 455, such as an electrical resistance of a component of the power storage device 455, an average electrical resistance (e.g., an arithmetic average electrical resistance) of one or more components of the power storage device 455, etc. A temperature related parameter refers to a parameter relating to a temperature associated with one or more components of the power storage device 455, such as a temperature of a component of the power storage device 455, an average temperature (e.g., an arithmetic average temperature) of one or more components of the power storage device 455, etc.

Values of the one or more parameters of the power storage device 455 may be detected using, for example, one or more sensors (e.g., at least one voltage sensor, at least one current sensor, at least one electrical resistance sensor, at least one temperature sensor, etc.). Exemplary voltage sensors may include a suspected transformer, a Hall voltage sensor, etc. Exemplary current sensors may include a Hall current sensor, a Rogowski current sensor, a fiber-optic current sensor, etc. Exemplary electrical resistance sensors may include a photoresistor sensor, a thermistor sensor, etc. Exemplary temperature sensors may include a mercurial thermometer, an infrared thermometer, etc.

The monitoring device 475 may process or analyze the values of the one or more parameters so as to determine the status of the power storage device 455. In some embodiments, the monitoring device 475 may determine whether one or more preset conditions associated with the status of the power storage device 455 are satisfied based on the values of the one or more parameters. If all of the one or more preset conditions are satisfied, the monitoring device 475 may determine that the power storage device 455 is in a normal status, and the process 900 may proceed to 930. The normal status may indicate that the power storage device is capable of providing power for the scanning apparatus 110 normally. If at least one of the one or more preset conditions is not satisfied, the monitoring device 475 may determine that the power storage device 455 is in an abnormal status, and the process 900 may proceed to 925. The abnormal status may indicate that the power storage device 455 may have a failure such as an open circuit, a short circuit, an abrupt temperature rise, etc. The one or more preset conditions may be set by a user, according to default settings of the automated scanning system 100, etc. Merey for illustration, the one or more preset conditions may include that a value of a voltage related parameter (e.g., an average voltage of one or more components of the power storage device 455) is below a corresponding voltage threshold, a value of a current related parameter (e.g., an average current of one or more components of the power storage device 455) is below a corresponding current threshold, a value of an electrical resistance related parameter (e.g., an average electrical resistance of one or more components of the power storage device 455) is below a corresponding electrical resistance threshold, a value of a temperature related parameter (e.g., an average temperature of one or more components of the power storage device 455) is below a corresponding temperature threshold, etc.

In 925, the power module 450 (e.g., the monitoring device 475) may disconnect a current path from the power source to the power storage device and generate failure information of the power storage device.

If at least one of the one or more preset conditions is not satisfied, the monitoring device 475 may determine that the power storage device is in an abnormal status. In this case, the monitoring device 475 may disconnect the current path from the power source to the power storage device 455 immediately to avoid further damages to the power storage device 455. The monitoring device 475 may also generate failure information of the power storage device 455. The failure information may include, for example, basic information of the power storage device 455 (e.g., a count of battery cells of the power storage device 455, a count of historical charging cycles, etc.), the values of the one or more parameters of the power storage device 455, the one or more preset conditions, potential failures of the power storage device 455, recommended approaches to deal with the potential failures, etc. The failure information may be transmitted to the terminal device 140 and/or displayed on the interface (e.g., a screen) set on the scanning apparatus 110.

In some embodiments, the power storage device 455 may need to be examined and/or repaired. For example, the power storage device 455 may be discharged using an ultra-fast discharge device (UFDD). Each component of the power storage device 455 may be examined so as to identify one or more damaged components. In some embodiments, the damaged components may be replaced or repaired.

In 930, the power module 450 (e.g., the monitoring device 475) may terminate the charging of the power storage device if the power storage device is fully charged.

If all of the one or more preset conditions are satisfied, the monitoring device 475 may determine that the power storage device 455 is in a normal status. In this case, the monitoring device 475 may control the charging circuit 470 to continue the charging of the power storage device 455 until the power storage device 455 is fully charged.

FIGS. 10A and 10B are schematic diagrams illustrating exemplary configurations of a wireless power receiver and a plurality of wireless power transmitters for wireless charging of a scanning apparatus according to some embodiments of the present disclosure. The scanning apparatus 110 may be implemented as a specific embodiment of the scanning apparatus 110 as illustrated in FIG. 1. As illustrated in FIGS. 10A and 10B, the scanning apparatus 110 may include a main body 1010. A detecting region (also referred as bore) 1012 may be configured in the main body 1010 for positioning and scanning a target portion of a subject. The scanning apparatus 110 may include a driving device that drives the scanning apparatus 110 to move to a position (e.g., a charging area). The driving device may include, for example, a driver (not shown) and a plurality of wheels 1020. The scanning apparatus 110 may further include a power storage device (not shown) and a wireless power receiver 1030. The power storage device may provide power for the scanning of the target portion and/or the moving of the scanning apparatus 110. The power storage device may be charged through the wireless power receiver 1030.

The scanning apparatus 110 (e.g., the power storage device thereof) may be charged at a charging station. The charging station may be a contactless charging station. The contactless charging station may include a plurality of wireless power transmitters 1040. The plurality of wireless power transmitters 1040 may be connected to an electricity grid 1050. For example, the plurality of wireless power transmitters 1040 may be connected in parallel and further connected to the electricity grid 1050.

The plurality of wireless power transmitters 1040 may be set in a transmitting area 1060. The transmitting area 1060 may have a circular shape, a rectangular shape, a square shape, or the like. In some embodiments, the transmitting area 1060 may have a shape of a rectangular shape. The plurality of wireless power transmitters 1040 may be arranged in a shape (e.g., a circle, a rectangle, a square, etc.) in the transmitting area 1060. For instance, the plurality of wireless power transmitters 1040 including nine wireless power transmitters may be arranged in a shape of a square having three rows and three columns.

When the scanning apparatus 110 is located in a charging area 1070, the wireless power receiver 1030 may be operably connected to one of the plurality of wireless power transmitters 1040 wirelessly to facilitate the wireless charging of the power storage device of the scanning apparatus 110. In some embodiments, the transmitting area 1060 may be within the charging area 1070. Merely for illustration, as illustrated in FIG. 10A, the transmitting area 1060 may be set on the ground in the charging area 1070. Alternatively, the transmitting area 1060 may be set in a plane at an angle with the charging area 1070. Merely for illustration, as illustrated in FIG. 10B, the transmitting area 1060 may be set in a wall. The wall may be in the Y-Z plane of the coordinate system 1080. The charging area 1070 may be in the X-Y plane of the coordinate system 1080. The transmitting area 1060 may be set in a plane perpendicular to the charging area 1070.

In some embodiments, a distance between the wireless power receiver 1030 (e.g., a center of a power receiving coil of the wireless power receiver 1030) and each of the plurality of wireless power transmitters 1040 (e.g., a center of a power transmitting coil of each of the plurality of wireless power transmitters 1040) may be determined. A shortest distance and a wireless power transmitter 1040 corresponding to the shortest distance may be identified. The wireless power receiver 1030 may be operably connected to the wireless power transmitter 1040 corresponding to the shortest distance wirelessly.

In some embodiments, when the wireless power receiver 1030 is operably connected to the wireless power transmitter 1040 corresponding to the shortest distance wirelessly, the power receiving coil of the wireless power receiver 1030 may be substantially aligned with the power transmitting coil of the wireless power transmitter 1040 so as to achieve a highest efficiency of the wireless charging and improve a stability of a charging current induced in the power receiving coil of the wireless power receiver 1030. In some embodiments, the center of the power receiving coil of the wireless power receiver 1030 may be substantially aligned with the center of the power transmitting coil of the wireless power transmitter 1040. Merely by way of example, both the power receiving coil and the power transmitting coil may be wound coils having annular cross-sections. Axes of the power receiving coil and the power transmitting coil may be substantially coincident.

In some embodiments, the scanning apparatus 110 may further include a coil adjustment device (not shown in the figure). If the power receiving coil is not aligned with the power transmitting coil, the coil adjustment device may drive the power receiving coil to move to be aligned with the power transmitting coil. In some embodiments, a variation of magnetic flux in the power receiving coil may be detected using a magnetic flux detection device. The magnetic flux detection device may be or include, for example, a Helmholtz coil. The variation of magnetic flux in the power receiving coil may be close to zero when the power receiving coil is substantially aligned with the power transmitting coil. In some embodiments, a magnetic field intensity in the power receiving coil may be detected using a magnetic field intensity detection device. The magnetic field intensity detection device may be or include, for example, a Hall element. The magnetic field intensity in the power receiving coil may have a largest value when the power receiving coil is substantially aligned with the power transmitting coil. The coil adjustment device may drive the power receiving coil to move to be aligned with the power transmitting coil according to the variation of the magnetic flux and/or the magnetic field intensity in the power receiving coil.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PUP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or parameters used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired parameters sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system, comprising:
at least one storage medium including a set of instructions; and
at least one processor configured to communicate with the at least one storage medium, wherein when executing the set of instructions, the at least one processor is configured to direct the system to perform operations including:
obtaining region information of a region;
determining, based on the region information, a route from a start position of a scanning apparatus to a target position;
causing the scanning apparatus to move to the target position along the route;
identifying a target portion of a subject based on image data obtained from at least one optical device set on the scanning apparatus; and
causing the scanning apparatus to scan the target portion of the subject.

2. The system of claim 1, wherein the obtaining region information of a region includes:
obtaining sensing data from one or more sensors set on the scanning apparatus; and
generating, based on the sensing data, the region information of the region.

3. The system of claim 2, wherein the one or more sensors include at least one distance sensor and at least one second optical device.

4. The system of claim 3, wherein the region information includes map data of the region.

5. The system of claim 4, wherein
the at least one distance sensor includes a first distance sensor and a second distance sensor, and
the generating, based on the sensing data, region information of a region includes:
determining environmental information of the region based on sensing data from the first distance sensor;
determining first supplementary information of the region based on sensing data from the at least one second optical device;
determining second supplementary information of the region based on sensing data from the second distance sensor; and
generating the map data of the region based on the environmental information, the first supplementary information, and the second supplementary information.

6. The system of claim 3, wherein the region information further includes route condition information.

7. The system of claim 6, wherein the generating, based on the sensing data, region information of a region includes:
identifying moving objects in the region based on the sensing data from the at least one second optical device;
determining movement statuses of the moving objects; and
generating the route condition information based on the movement statuses of the moving objects.

8. The system of claim 6, wherein the causing the scanning apparatus to move to the target position along the route includes:
determining a moving speed of the scanning apparatus based on the route condition information; and
causing the scanning apparatus to move to the target position along the route at the determined moving speed.

9. The system of claim 1, wherein the region information includes map data of the region.

10. The system of claim 1, wherein the determining, based on the region information, a route from a start position of a scanning apparatus to a target position where a subject is located includes:
obtaining a moving model; and
determining the route from the start position of the scanning apparatus to the target position by inputting the region information, the start position, and the target position into the moving model.

11. The system of claim 10, wherein the moving model is a machine learning model.

12. The system of claim 1, wherein the causing the scanning apparatus to scan the target portion of the subject includes:
obtaining at least one of a position difference or a direction difference between the target portion and the scanning apparatus; and
causing the scanning apparatus to adjust at least one of a position or a direction of the scanning apparatus according to at least one of the position difference or the direction difference.

13. The system of claim 1, the operations further including:
determining a moving mode of the scanning apparatus according to at least one of the region information or user instructions.

14. The system of claim 13, wherein the determination of the route from the start position to the target position and the moving of the scanning apparatus to the target position along the route are in accordance with the moving mode.

15. The system of claim 1, the operations further including:
causing the scanning apparatus to move to a charging area for charging the scanning apparatus at a contactless charging station or a contact charging station in the charging area.

16. The system of claim 15, wherein
the contactless charging station includes a plurality of wireless power transmitters set in a transmitting area, the plurality of wireless power transmitters being electrically connected to a power source;
the scanning apparatus includes a wireless power receiver and a power storage device, the wireless power receiver being electrically connected to the power storage device; and
the wireless power receiver is operably connected to one of the plurality of wireless power transmitters wirelessly to facilitate the charging of the power storage device of the scanning apparatus when the scanning apparatus is located in the charging area.

17. The system of claim 16, wherein
the one of the plurality of wireless power transmitters includes a power transmitting coil,
the wireless power receiver includes a power receiving coil, and
the power transmitting coil is aligned with the power receiving coil when the wireless power receiver is operably connected to the one of the plurality of wireless power transmitters wirelessly.

18-21. (canceled)

22. The system of claim 1, wherein the operations further include:
determining an updated route from an intermediate position between the start position and the target position to the target position.

23. (canceled)

24. A system, comprising:
a scanning apparatus, configured to scan a target portion of a subject;
at least one optical device, configured to generate image data including a representation of the subject;
at least one processor, configured to:
obtain region information of a region;
determine, based on the region information, a route from a start position of the scanning apparatus to a target position;
cause the scanning apparatus to move to the target position along the route; and
identify the target portion of the subject based on the image data including the representation of the subject.

25-48. (canceled)

49. A method implemented on a computing device having a processor and a computer-readable storage device, the method comprising:
obtaining region information of a region;
determining, based on the region information, a route from a start position of a scanning apparatus to a target position;
causing the scanning apparatus to move to the target position along the route;
identifying a target portion of a subject based on image data obtained from at least one optical device set on the scanning apparatus; and
causing the scanning apparatus to scan the target portion of the subject.

50-72. (canceled)
